(19) United States
(12) Patent Application Publication
(10) Pub. No.: US 2013/0085133 A1
(43) Pub. Date: Apr. 4, 2013

(54) ANTI-VIRAL TREATMENT AND ASSAY TO SCREENFOR ANTI-VIRAL AGENT

(71) Applicants: Bill Severson, Birmingham, AL (US); Dong Hong Chung, Prospect, KY (US); Colleen B. Jonsson, Louisville, KY (US); Ellie Lucile White, Birmingham, AL (US); Lynn Rasmussen, Odenville, AL (US); Clinton Boykin Maddox, Hoover, AL (US); Subramaniam Ananthan, Birmingham, AL (US); Ashish K. Pathak, Birmingham, AL (US); Joseph A. Maddry, Birmingham, AL (US)

(72) Inventors: Bill Severson, Birmingham, AL (US); Dong Hong Chung, Prospect, KY (US); Colleen B. Jonsson, Louisville, KY (US); Ellie Lucile White, Birmingham, AL (US); Lynn Rasmussen, Odenville, AL (US); Clinton Boykin Maddox, Hoover, AL (US); Subramaniam Ananthan, Birmingham, AL (US); Ashish K. Pathak, Birmingham, AL (US); Joseph A. Maddry, Birmingham, AL (US)

(73) Assignee: Sourthern Research Institute Office of Commercialization and Intellectual Prop., Birmingham, AL (US)

(21) Appl. No.: 13/577,592

(22) PCT Filed: Feb. 8, 2011
(86) PCT No.: PCT/US11/23986

§ 371 Date: Dec. 19, 2012

Related U.S. Application Data
(60) Provisional application No. 61/302,383, filed on Feb. 8, 2010.

(51) Int. Cl. C07H 19/12; C07D 471/14; C12Q 1/02; C07D 471/04

(52) U.S. Cl. CPC C07H 19/12; C07D 471/04; C07D 471/14; C12Q 1/025
USPC 514/214.02; 514/264.11; 514/43; 435/5; 506/10

(57) ABSTRACT

The present disclosure relates to novel compounds of formulas (1) through (19) and to a method for treating humans infected with a virus including various respiratory viruses such as members of the Paramyxoviridae family (respiratory syncytial virus (RSV), human metapneumovirus (HMPV), human parainfluenza virus (HPIV), measles virus, and mumps virus) with a compound of formulas (1) through (19). The present disclosure also relates to a cytopathic effect (CPE)-based assay that will assess virus-induced CPE for screening of compounds for treating viral diseases or inhibiting a virus.

### TECHNICAL FIELD

The present disclosure relates to a method for treating humans infected with a virus including various respiratory viruses such as members of the Paramyxoviridae family (respiratory syncytial virus (RSV), human metapneumovirus (HMPV), human parainfluenza virus (HPIV), measles virus, and mumps virus) with a compound of formulas (1) through (19). The applications of this disclosure also include those situations in which preventing virus-induced cytopathic effect (CPE) can result in the protection against infections. The present disclosure also relates to those compounds of this disclosure that are novel. The present disclosure also relates to a CPE-based assay that will assess virus-induced CPE for screening of compounds for treating viral diseases or inhibiting a virus.

### BACKGROUND OF DISCLOSURE

Currently, there are no commercially available vaccines to protect humans against respiratory syncytial virus (RSV). RSV is associated with substantial morbidity and mortality and is the most common cause of bronchiolitis and pneumonia among infants and children under one year of age. Nevertheless, severe lower respiratory tract disease may occur at any age, especially among the elderly or among those with compromised cardiac, pulmonary, or immune systems. The existing therapies for the acute infection are Ribavirin and the prophylactic humanized monoclonal antibody (Synagis® from MedImmune) that is limited to use in high risk pediatric patients. The economic impact of RSV infections due to hospitalizations and indirect medical costs is >$650 million annually. The current health burden of RSV infections has increased effort towards the discovery and development of antivirals and vaccines for the treatment of the disease.

The hosts or patients treated according to this disclosure include humans.

Drug development efforts in this therapeutic area have been unable to make any significant progress due to a lack of assays suitable for High Throughput Screening. Virus instability has made any work with RSV difficult and High Throughput Screening problematic, therefore progress has been slow in drug development. What is disclosed here is a novel strategy that circumvents previous problems and has allowed the development of a HTS assay for drug development.

### SUMMARY OF DISCLOSURE

The present disclosure also relates to a CPE-based assay that will assess virus-induced CPE for screening of compounds for treating viral diseases or inhibiting a virus. In particular, the present disclosure is concerned with a method for screening for compounds for use as an anti-viral agent against a virus which comprises obtaining frozen cells infected with said virus, thawing said infected cells and mixing said infected cells with uninfected cells of the same type as the infected cells, contacting the mixture of said infected cells and uninfected cells with a compound to be screened and determining the viability of said cells.

Another aspect of the present disclosure relates to a method for treating a human infected with a virus by administering to said human an effective amount of at least one compound represented by the formulas 1-19 below:

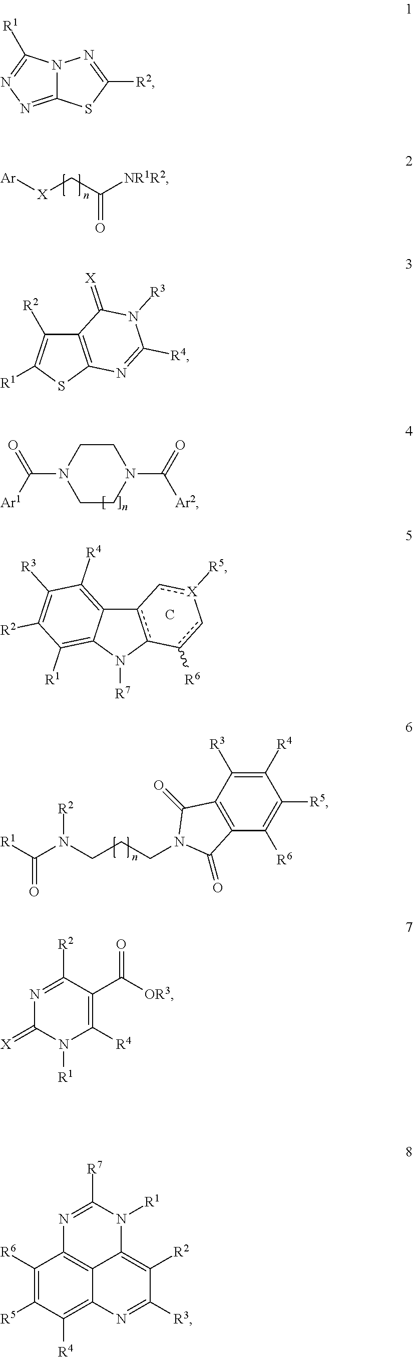

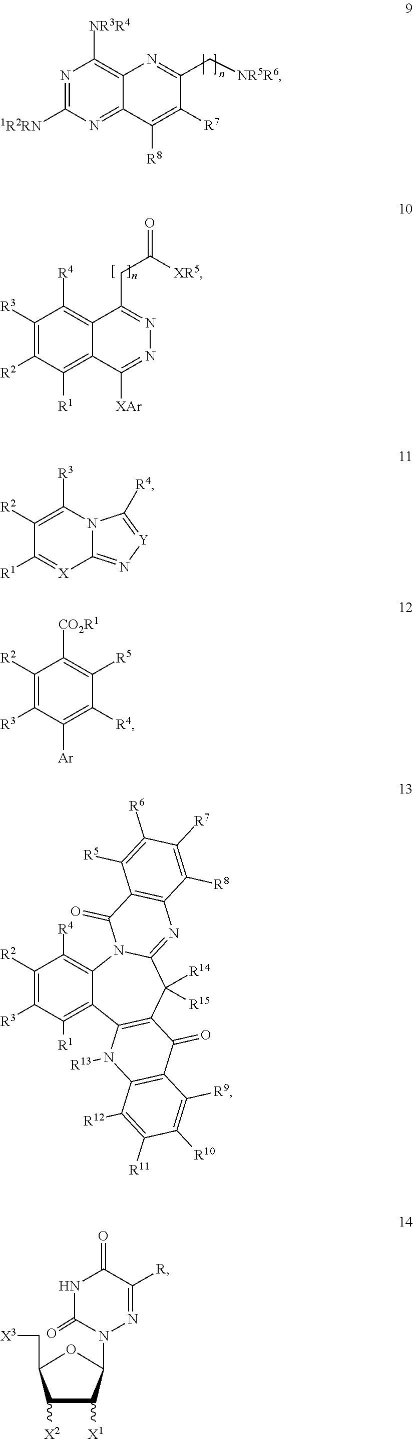

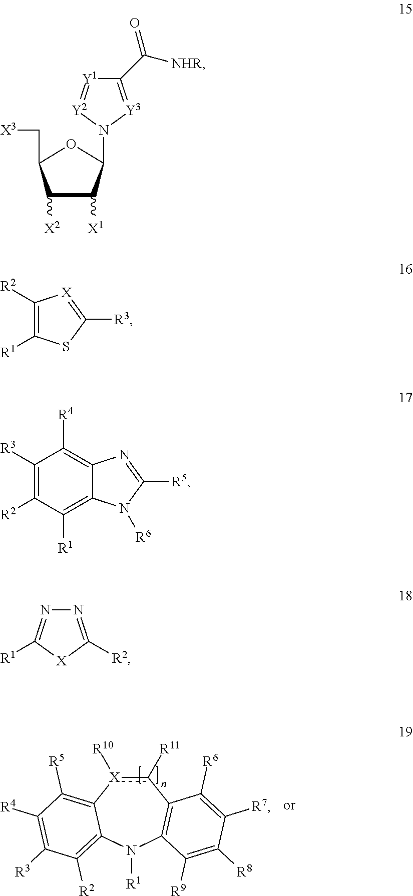

a pharmaceutically acceptable salt thereof, a solvate thereof, or a prodrug thereof; wherein in formula 1,

each R¹ and R² is independently chosen from the group consisting of hydrogen, substituted or unsubstituted alkyl, substituted or unsubstituted alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate ester, carboxamido, amino, and mono- or di-substituted amino;

wherein in formula 2,

n=1, 2, or 3;

X is chosen from sulfur, oxygen, and substituted or unsubstituted nitrogen;

Ar is chosen from the group consisting of substituted or unsubstituted aryl, substituted or unsubstituted arylalkyl, substituted or unsubstituted heteroaryl, substituted or unsubstituted cycloalkyl, or substituted or unsubstituted heterocyclic;

R¹ and R² are independently chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, and acyl;

wherein in formula 3,

X is chosen from oxygen or substituted nitrogen,

R¹, R², and R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxyl, carboxylate esters, and carboxamido;

wherein in formula 4,

n=0, 1, or 2;

Ar¹ may be attached directly to the nitrogen atom without the linking carbonyl group and if both carbonyl groups are present, then one of the nitrogen atoms may be replaced by carbon;

Ar¹ and Ar² are each independently chosen from the group consisting of substituted or unsubstituted aryl, substituted or unsubstituted arylalkyl, substituted or unsubstituted heteroaryl, substituted or unsubstituted cycloalkyl, and substituted or unsubstituted heterocyclic;

wherein in formula 5,

the ring designated C is optional but if present may be saturated or partially or fully unsaturated;

if ring C is absent, then the pyrrole ring may optionally have one or two additional substituents instead;

X is substituted or unsubstituted carbon or substituted or unsubstituted nitrogen;

R¹-R⁴, R⁶, and R⁵ when X═C, are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

when ring C is absent, then the one or two additional substituents on the pyrrole ring, if present, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R⁵ when X═N, and R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl; carboxyl, carboxylate esters, and carboxamido;

wherein in formula 6,

n=1, 2, or 3;

R¹ is chosen from the group consisting of substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, alkylthio, amino, and mono- or di-substituted amino;

R² is chosen from the group consisting of hydrogen, substituted or unsubstituted alkyl, substituted or unsubstituted unsaturated alkyl, substituted or unsubstituted aryl; substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R³-R⁶ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

wherein in formula 7,

X is chosen from oxygen and sulfur;

R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic including glycosyl rings, acyl, carboxylate esters, and carboxamido;

R² and R⁴ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;

wherein in formula 8,

R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R² through R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

wherein in formula 9,

n=0, 1, or 2;

R¹ through R⁶ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R⁷ and R⁸ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

wherein in formula 10,

n=0, 1, or 2;

X is chosen from the group consisting of N, O, and S;

R¹ through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R⁵ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic;

wherein in formula 11,

X and Y are independently chosen from C and N;

R¹ through R⁴ are independently chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

if X═C or Y═C, then either may independently be substituted by additional R moieties chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

wherein in formula 12,

R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;

R² through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic;

wherein in formula 13,

R¹ through R¹², R¹⁴ and R¹⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R¹³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

wherein in formula 14,

X¹, X², and X³ are independently chosen from the group consisting of O, S, N, C, and halogen;

whenever any of X¹, X², and X³ is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;

the R group is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

wherein in formula 15,

X¹, X², and X³ are independently chosen from the group consisting of O, S, N, C, and halogen;

whenever any of these groups is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;

Y¹, Y², and Y³ are independently chosen from the group consisting of N and C;

R is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

wherein in formula 16,

X is chosen from the group consisting of N and C;

R¹, R², and R³ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

if X═C, it may be further independently substituted by a group from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

wherein in formula 17,

R¹ through R⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R⁶ is chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

wherein in formula 18,

X is chosen from the group consisting of O, S, and N;

R¹ and R² are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

if X═N, then it is substituted by a moiety chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

wherein in formula 19,

n=0 or 1;

if n=0, the substituent R¹¹ does not occur;

X is chosen from the group consisting of C, O, S, and N;

if X═C or X═N, then it may substituted by a group R¹⁰;

if X═O or X═S, then substituent R¹⁰ does not occur;

if n=1 and X═C or N, then the bond to X internal to the seven-membered ring may optionally be unsaturated;

R¹ and R¹⁰ in the case X═N, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R² through R⁹, R¹⁰ in the case X═C, and substituent R¹¹ in the case n=1, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.

A still further aspect of the present disclosure relates to preventing virus-induced cytopathic effect (CPE) in a human patient for protection against infections which comprises administering to said human an effective amount of at least one compound represented by the formulas 1-19 as disclosed herein above.

The present disclosure is also concerned with those compounds defined above that are novel.

Still other objects and advantages of the present disclosure will become readily apparent by those skilled in the art from the following detailed description, wherein it is shown and described preferred embodiments, simply by way of illustration of the best mode contemplated. As will be realized the disclosure is capable of other and different embodiments, and its several details are capable of modifications in various obvious respects, without departing from the disclosure. Accordingly, the description is to be regarded as illustrative in nature and not as restrictive.

### SUMMARY OF FIGURES

FIG. 1 is a Venn diagram of activities of tested compounds from the assay according to the present disclosure and from the diluted virus assay.

FIG. 2 is a MSR plot of potency ratio vs. geometric means for the EC₅₀ analysis

FIG. 3 is a scatter plot and analysis for the EC₅₀ analysis.

FIG. 4 is a MSR plot of potency ratio vs. geometric means for the EC₉₀ analysis.

FIG. 5 is a scatter plot and analysis for the EC₉₀ analysis.

### BEST AND VARIOUS MODES

The compounds employed according to the present disclosure are represented by the formulas 1-19 below:

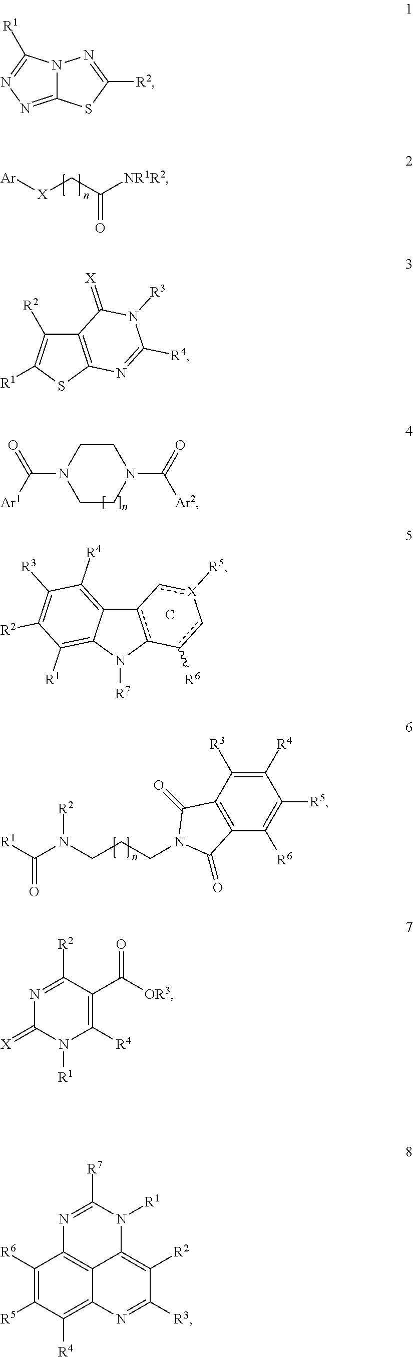

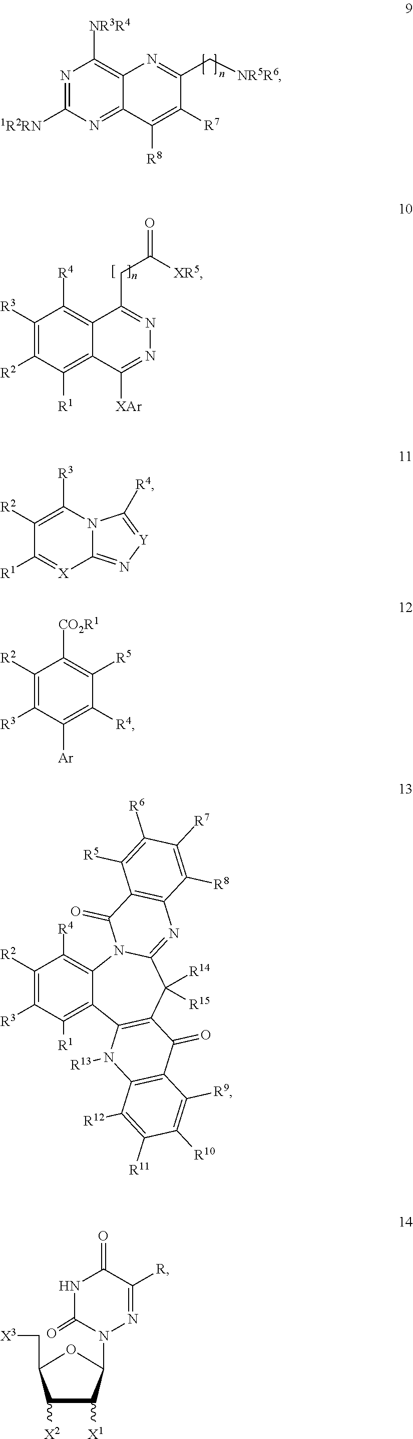

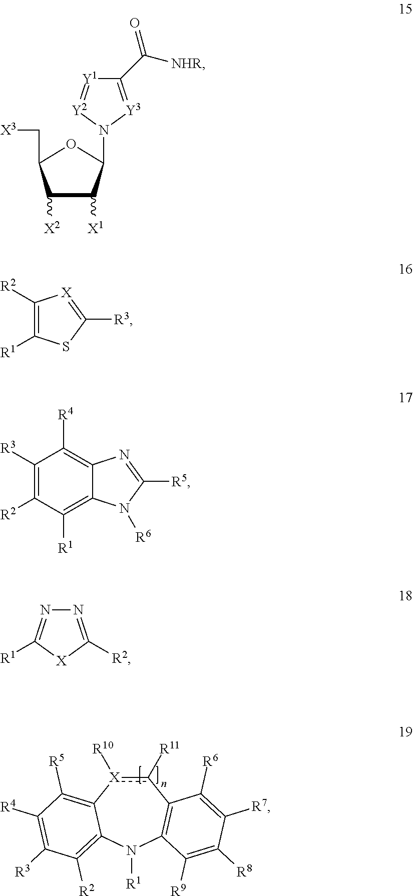

optionally in their stereoisomerically pure form, a pharmaceutically acceptable salt thereof, a solvate thereof, a prodrug thereof, and mixtures thereof.

In formula 1, each R¹ and R² is independently chosen from the group consisting of hydrogen, substituted or unsubstituted alkyl, substituted or unsubstituted alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate ester, carboxamido, amino, and mono- or di-substituted amino.

Examples of this class of compounds include AB00369924 and AB00370063.

In formula 2, n=1, 2, or 3;

X is chosen from sulfur, oxygen, and substituted or unsubstituted nitrogen;

Ar is chosen from the group consisting of substituted or unsubstituted aryl, substituted or unsubstituted arylalkyl, substituted or unsubstituted heteroaryl, substituted or unsubstituted cycloalkyl, or substituted or unsubstituted heterocyclic;

R¹ and R² are independently chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, and acyl.

Examples of this class include AB00345103, AB00341850, AB00756405, AB00358122, AB00289457, AB00306112, AB00368252, and AB00293237.

In formula 3, X is chosen from oxygen or substituted nitrogen;

R¹, R², and R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxyl, carboxylate esters, and carboxamido.

Examples of this class include AB00298501, AB00297372, AB00372085, AB00728757, AB00276706, and AB00796970.

In formula 4, n=0, 1, or 2.;

Ar¹ may be attached directly to the nitrogen atom without the linking carbonyl group and if both carbonyl groups are present, then one of the nitrogen atoms may be replaced by carbon;

Ar¹ and Ar² are each independently chosen from the group consisting of substituted or unsubstituted aryl, substituted or unsubstituted arylalkyl, substituted or unsubstituted heteroaryl, substituted or unsubstituted cycloalkyl, and substituted or unsubstituted heterocyclic.

Examples of this class include AB00312102, AB00311948, AB00315446, AB00319534, AB00298665, AB00299386, AB00317214, AB00284323, AB00299547, and AB00309842.

In formula 5, the ring designated C is optional but if present may be saturated or partially or fully unsaturated;

if ring C is absent, then the pyrrole ring may optionally have one or two additional substituents instead;

X is substituted or unsubstituted carbon or substituted or unsubstituted nitrogen;

R¹-R⁴, R⁶, and R⁵ when X═C, are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

when ring C is absent, then the one or two additional substituents on the pyrrole ring, if present, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R⁵ when X═N, and R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl; carboxyl, carboxylate esters, and carboxamido.

Examples of this class include AB00722846, AB00275559, AB00705281, AB00279132, and AB00291074.

In formula 6, n=1, 2, or 3;

R¹ is chosen from the group consisting of substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, alkylthio, amino, and mono- or di-substituted amino;

R² is chosen from the group consisting of hydrogen, substituted or unsubstituted alkyl, substituted or unsubstituted unsaturated alkyl, substituted or unsubstituted aryl; substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R³-R⁶ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

Examples of this class include AB00302018, AB00300194, AB00302020, AB00301782, and AB00301184.

In formula 7, X is chosen from oxygen and sulfur;

R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic including glycosyl rings, acyl, carboxylate esters, and carboxamido;

R² and R⁴ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;

Examples of this class include AB00355044, AB00318401, AB00319298, AB00318114, and AB00316243.

In formula 8,

R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R² through R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.

Examples of this class include AB00280244.

In formula 9, n=0, 1, or 2;

R¹ through R⁶ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R⁷ and R⁸ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

Examples of this class include SRI-10531.

In formula 10, n=0, 1, or 2;

X is chosen from the group consisting of N, O, and S;

R¹ through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R⁵ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic.

Examples of this class include AB00285095.

In formula 11, X and Y are independently chosen from C and N;

R¹ through R⁴ are independently chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio,; carboxyl, carboxylate esters, carboxamido, amino, and mono- or di-substituted amino;

if X═C or Y═C, then either may independently be substituted by additional R moieties chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.

Examples of this class include AB00292655, AB00877180, and AB00356285.

In formula 12, R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;

R² through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic;

Examples of this class include AB00275199.

In formula 13, R¹ through R¹², R¹⁴ and R¹⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R¹³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido.

Examples of this class include AB00700560.

In formula 14, X¹, X², and X³ are independently chosen from the group consisting of O, S, N, C, and halogen;

whenever any of X¹, X², and X³ is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;

the R group is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.

Examples of this class include SRI-7958 and AB00174524.

In formula 15, X¹, X², and X³ are independently chosen from the group consisting of O, S, N, C, and halogen;

whenever any of these groups is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;

Y¹, Y², and Y³ are independently chosen from the group consisting of N and C;

R is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido

Examples of this class include AB00430481.

In formula 16, X is chosen from the group consisting of N and C;

R¹, R², and R³ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

if X═C, it may be further independently substituted by a group from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.

Examples of this class include AB00310808, AB00361531, AB00310910, AB00313042, AB00313952, AB00355020, AB00367930, AB00310739, AB00309154, AB00309859, AB00299380, AB00747970, and AB00372085.

In formula 17, R¹ through R⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

R⁶ is chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido.

Examples of this class include AB00369924, AB00627942, AB00296415, AB00308659, AB00321587, AB00709376, AB00358081, AB00368222, AB00342188, AB00369934, AB00348716, and AB00364575.

In formula 18, X is chosen from the group consisting of O, S, and N;

R¹ and R² are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;

if X═N, then it is substituted by a moiety chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido.

Examples of this class include AB00724801, AB00725275, AB00430481, AB00767910, AB00292171, AB00356597, AB00372085, AB00358926, and AB00370949.

In formula 19,

n=0 or 1;

if n=0, the substituent R¹¹ does not occur;

X is chosen from the group consisting of C, O, S, and N;

if X═C or X═N, then it may substituted by a group R¹⁰;

if X═O or X═S, then substituent R¹⁰ does not occur;

if n=1 and X═C or N, then the bond to X internal to the seven-membered ring may optionally be unsaturated;

R¹ and R¹⁰ in the case X═N, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;

R² through R⁹, R¹⁰ in the case X═C, and substituent R¹¹ in the case n=1, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.

Examples of this class include AB00139215, AB00276381, and AB00370949.

Listed below are definitions of various terms used to describe this invention. These definitions apply to the terms as they are used throughout this specification, unless otherwise limited in specific instances, either individually or as part of a larger group.

Typical aliphatic acyl groups contain 1 to 6 carbon atoms and include formyl, acetyl and propionyl.

Typical aromatic acyl groups include unsubstituted and alkyl substituted aromatic groups containing 7 to 10 carbon atoms in the aromatic ring. When substituted the alkyl group typically contains 1-6 carbon atoms. Typical aromatic acyl groups include benzoyl and para-toluoyl.

The term “alkyl” refers to straight or branched chain unsubstituted hydrocarbon groups of typically 1 to 22 carbon atoms, more typically 1 to 8 carbon atoms, and even more typically 1 to 4 carbon atoms.

Examples of suitable alkyl groups include methyl, ethyl and propyl. Examples of branched alkyl groups include isopropyl and t-butyl.

The term “cycloalkyl” refers to optionally substituted, saturated cyclic hydrocarbon ring systems, preferably containing 1 to 3 rings and 3 to 7 carbons per ring which may be further fused with an unsaturated C₃-C₇ carbocyclic ring. Exemplary groups include cyclopropyl, cyclobutyl, cyclopentyl, cyclohexyl, cyclopropylmethyl cycloheptyl, cyclooctyl, cyclodecyl, cyclododecyl and adamantyl. Exemplary substituents include one or more alkyl groups as described above, or one or more groups described above as alkyl substituents.

Examples of unsaturated alkyl groups include ethynyl, cyclopentenyl, and allyl. Examples of substituted alkyl groups include 2-methoxyethyl, 2,2,2-trifluoroethyl, and 2-diethylaminocyclopentenyl.

The alkoxy group typically contains 1 to 6 carbon atoms. Suitable alkoxy groups typically contain 1-6 carbon atoms and include methoxy, ethoxy, propoxy and butoxy.

Suitable haloalkyl groups typically contain 1-6 carbon atoms and can be straight or branched chain and include Cl, Br, F or I, substituted alkyl groups including the above specifically disclosed alkyl groups.

Suitable alkenyl groups typically contain 2-6 carbon atoms and include ethenyl and propenyl.

Suitable haloalkenyl groups typically contain 1-6 carbon atoms and include Cl, Br F or I, substituted alkenyl groups including the above specifically disclosed alkenyl groups.

Suitable alkynyl groups typically contain 1-6 carbon atoms and include ethynyl and propynyl.

Suitable monoalkylamino groups contain 1-6 carbon atoms and include monomethylamino, monoethylamino, mono-isopropylamino, mono-n-propylamino, mono-isobutyl-amino, mono-n-butylamino and mono-n-hexylamino. The alkyl moiety can be straight or branched chain.

Suitable dialkylamino groups contain 1-6 carbon atoms in each alkyl group. The alkyl groups can be the same or different and can be straight or branched chain. Examples of some suitable groups are dimethylamino, diethylamino, ethylmethylamino, dipropylamino, dibutylamino, dipentylamino, dihexylamino, methylpentylamino, ethylpropylamino and ethylhexylamino.

Examples of halo groups are Cl, F, Br and I.

The term “aryl” refers to monocyclic or polycyclic aromatic hydrocarbon groups having 6 to 14 carbon atoms in the ring portion, such as phenyl, naphthyl, biphenyl, and diphenyl groups, each of which may be substituted such as with a halo or alkyl group.

The term “aralkyl” or “alkylaryl” refers to an aryl group bonded directly through an alkyl group, such as benzyl or phenethyl.

The term “heteroaryl”, refers to an optionally substituted, unsaturated aromatic cyclic group, for example, which is a 5 to 7 membered monocyclic, 7 to 11 membered bicyclic, or 10 to 15 membered tricyclic ring system, which has at least one heteroatom and at least one carbon atom in the ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. Examples of heteroaryl groups are pyridyl, imidazolyl, oxazolyl, thiazolyl, isothiazolyl, furyl, thienyl and indolyl.

The terms “heterocycle”, “heterocyclic” and “heterocyclo” refer to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom and at least one carbon atom in the ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom. Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, isopyrrole, 1,2,3-triazole, 1,2,4-triazole, oxazole, thiazole, pyrimidine, aziridines, thiazole, 1,2,3-oxadiazole, thiazine, pyrrolidine, oxaziranes, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N6-alkylpurines, N6-benzylpurine, N6-halopurine, N6-vinypurine, N6-acetylenic purine, N6-acyl purine, N6-hydroxyalkyl purine, N6-thioalkyl purine, thymine, cytosine, 6-azapyrimidine, 2-mercaptopyrmidine, uracil, N5-alkyl-pyrimidines, N5-benzylpyrimidines, N5-halopyrimidines, N5-vinyl-pyrimidine, N5-acetylenic pyrimidine, N5-acyl pyrimidine, N5-hydroxyalkyl purine, and N6-thioalkyl purine, and isoxazolyl.

The heteroaromatic and heterocyclic moieties can be optionally substituted as described above for aryl, including substituted with one or more substituents selected from hydroxyl, amino, alkylamino, arylamino, alkoxy, aryloxy, alkyl, heterocycle, halo, carboxy, acyl, acyloxy, amido, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “carboxylate ester” (e.g., carboxylic acid ester) refers to a carboxy group —C(═O)OR′, wherein R′ is substituted or unsubstituted alkyl, substituted or unsubstituted alkenyl, substituted or unsubstituted alkynyl, substituted or unsubstituted carbocyclic, substituted or unsubstituted heterocyclic, substituted or unsubstituted aryl, or substituted or unsubstituted aralkyl.

When any of the above groups are substituted, unless stated otherwise, they are typically substituted with at least one member selected from the group consisting of alkyl, hydroxyl, alkoxy, amino, halo and halogenated alkyl. A typical halogenated alkyl is a fluoroalkyl such as trifluoromethyl.

The terms “effective amount” or “therapeutically effective amount” refer to an amount of the compound of the invention sufficient to provide a benefit in the treatment or prevention of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent

The term “treating” refers to relieving the disease, disorder, or condition, i.e., causing regression of the disease, disorder, and/or condition; preventing a disease, disorder, or condition from occurring in an animal that may be predisposed to the disease, disorder and/or condition, but has not yet been diagnosed as having it; and/or inhibiting the disease, disorder, or condition, i.e., arresting its development.

It is of course understood that the compounds of the present disclosure relate to all optical isomers and stereo-isomers at the various possible atoms of the molecule, unless specified otherwise.

The compounds according to this disclosure may form prodrugs at hydroxyl or amino functionalities using alkoxy, amino acids, etc. groups as the prodrug forming moieties. For instance, the hydroxymethyl position may form mono-, di- or triphosphates and again these phosphates can form prodrugs.

Preparations of such prodrug derivatives are discussed in various literature sources (examples are: Alexander et al., J. Med. Chem. 1988, 31, 318; Aligas-Martin et al., PCT WO pp/41531, p. 30). The nitrogen function converted in preparing these derivatives is one (or more) of the nitrogen atoms of a compound of the disclosure.

“Pharmaceutically acceptable salts” refer to derivatives of the disclosed compounds wherein the parent compound is modified by making acid or base salts thereof. The compounds of this disclosure form acid and base addition salts with a wide variety of organic and inorganic acids and bases and includes the physiologically acceptable salts which are often used in pharmaceutical chemistry. Such salts are also part of this disclosure. Typical inorganic acids used to form such salts include hydrochloric, hydrobromic, hydroiodic, nitric, sulfuric, phosphoric, hypophosphoric and the like. Salts derived from organic acids, such as aliphatic mono and dicarboxylic acids, phenyl substituted alkonic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, cabrate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

Bases commonly used for formation of salts include ammonium hydroxide and alkali and alkaline earth metal hydroxides, carbonates, as well as aliphatic and primary, secondary and tertiary amines, aliphatic diamines. Bases especially useful in the preparation of addition salts include sodium hydroxide, potassium hydroxide, ammonium hydroxide, potassium carbonate, methylamine, diethylamine, and ethylene diamine.

“Solvates” refers to the compound formed by the interaction of a solvent and a solute and includes hydrates. Solvates are usually crystalline solid adducts containing solvent molecules within the crystal structure, in either stoichiometric or non-stoichiometric proportions.

The term “comprising” (and its grammatical variations) as used herein is used in the inclusive sense of “having” or “including” and not in the exclusive sense of “consisting only of”. The terms “a” and “the” as used herein are understood to encompass the plural as well as the singular.

Following is a discussion of the protocols employed concerning the assay of according to the present disclosure.

In particular, a cytopathogenic based assay (CPE) to screen large compound libraries (>100,000 compounds) against respiratory syncytial virus (RSV) strain Long has been developed according to this disclosure. The assay measures RSV virus-induced CPE in HEp-2 cells using cell viability as the end point. An important aspect of the present disclosure that allowed the development of an HTS compatible assay was the use of virally infected frozen cells in place of the conventional infectious virus as the source of infectious material in the assay.

Cell Culture:

HEp-2 cells (ATCC CCL-23, American Tissue Culture Type) were maintained as adherent cell lines in Optimem 1 with 2 mM L-glutamine and 5% fetal bovine serum (FBS) at 37° C. in a humidified 5% CO₂ atmosphere. Cells were passaged as needed and harvested from flasks using 0.25% trypsin-EDTA.

Assay Media—Preparation of Complete DMEM/F12:

50 mL Pen/Strep/Glutamine (Gibco, Cat #10378) was added to four liters of room temperature DMEM/F12 (Sigma, Cat # D6434) and the pH adjusted to 7.5 using 1N NaOH. The medium was sterile filtered through a 0.2 um filter and 10 mL of HI-FBS was added per 500 mL of media.

Infectious Material—Frozen Infected Virus Cell Preparation:

Two vials of RSV (strain Long) containing 1×10⁷ pfu/mL was thawed using an Eppendorf thermomixer for 13 min at 15° C., with shaking at 350 rpm. Two mL of the virus stock was added to a T-225 flask containing 3.0×10⁸ HEp-2 cells in 30 mL Complete DMEM/F12. The cells were incubated for 18-20 h at 37° C., 5% CO₂, 90% relative humidity. The medium was aspirated and the cells washed with 10 mL PBS without Mg²⁺ or Ca²⁺. Cells were harvested from flasks using 0.25% trypsin-EDTA. The cells were resuspended in a freezing medium of 95% fetal calf serum and 5% DMSO to a final cell density of 2×10⁶ cells/mL. One mL aliquots of this virus infected cell suspension were dispensed to cryovials and cells were rate frozen to −80° C. Frozen infected cells were then transferred to −150° C. for long term storage.

### Assay Protocols:

Single Dose Compound Preparation:

For single dose screening, compounds or carrier control (DMSO) were diluted to 6× in Complete DMEM/F12 and 5 ul was dispensed to assay plates (3% DMSO or 60 uM compound in 3% DMSO).

Dose Response Compound Preparation:

Test compounds were evaluated by measuring their antiviral activity, cell toxicity, and selectivity. Test compounds were serially diluted in a plate to plate matrix or “stacked plate” matrix. All 320 compounds in a source plate were diluted together resulting in a 10 point dose response dilution series. It was visualized as a serial dilution series proceeding vertically through a stack of plates with the high dose plate on top and the low dose plate on the bottom. The final plate well concentration ranged from 50 uM to 0.097 uM for the Enamine compound library; 25 ug/mL to 0.048 ug/mL for the Chembridge compound library; 50 uM to 0.097 uM for the SRI Proprietary library; 25 uM to 0.048 uM for the Ole Miss library; 50 ug/mL to 0.097 ug/mL and 30 ug/mL to 0.058 ug/mL for the SRI Collaborator library and a final DMSO concentration of 0.5%.

Control Drug:

The positive control drug for this assay, ribavirin. Hruska, J. F., et al., Effects of ribavirin on respiratory syncytial virus in vitro. Antimicrob Agents Chemother, 1980. 17(5): p. 770-5. (#196066, MP Biomedicals, Solon, Ohio) was solubilized in DMSO. It was diluted and added to the assay plates as described for test compounds. Final concentration for ribavirin was 35 uM. All wells contained 0.5% DMSO.

Preparation of HEp-2 Cells:

Cells were harvested and resuspended to 80,000 cells per ml in Complete DMEM/F12.

Frozen infected HEp-2 Cells:

Cells were thawed in a room temperature water bath with gentle agitation. The tube was inverted 5-10. Cells were diluted to 80,000 cells per ml by adding the contents of the vial to 24 ml of cold (4° C.) media.

Assay Set Up:

Twenty five ul of uninfected HEp-2 cells were plated in the cell control wells. Frozen infected cells were combined with uninfected HEp-2 cells at a 1:100 ratio. Twenty five ul of the cell mixture was added to the virus control and compound wells. All cell plating was conducted using a Matrix WellMate and cells were maintained at room temperature with stirring during the plating process. The assay plates were incubated for six days at 37° C., 5% CO₂ and 90% relative humidity.

Endpoint Read:

Following the six day incubation period, the assay plates were equilibrated to room temperature for 30 min and an equal volume (30 μL) of Cell Titer-Glo reagent (Promega Inc.) was added to each well using a WellMate (Matrix, Hudson, N.H.) and plates were incubated for an additional 10 min at room temperature. At the end of the incubation, luminescence was measured using a Perkin Elmer Envision™ multi-label reader (PerkinElmer, Wellesley, Mass.) with an integration time of 0.1 s.

Data Analysis:

Data was analyzed using ActivityBase software (IDBS, Inc, Guilford, UK). thirty two control wells containing cells only and twenty four wells containing cells and virus were included on each assay plate and used to calculate Z′ value for each plate and to normalize the data on a per plate basis. The overall Z score for the campaign were 0.7. Results are reported as percent (%) CPE inhibition and were calculated using the following formula: % CPE inhibition=100*(Test Cmpd−Med Virus)/(Med Cells−Med Virus). Eight ribavirin positive control wells were included on each plate for quality control purposes, but were not used in Z′ calculations. Dose Response: Confirmation of active compounds were done in two formats, the CPE assay described above to evaluate antiviral activity and a cytotoxicity assay used to evaluate the toxicity of the compounds, Antiviral activity is described as percent CPE inhibition=100*((luminescence compound well−median luminescence virus control)/(median luminescence cell control−median luminescence virus control. In the toxicity assay, results are described as percent viability and the calculation is the same. 100% viability in a compound well would indicate 100% inhibition of viral CPE in the antiviral assay or no toxicity in the cytotoxicity assay. The Z factor values were calculated from 1 minus (3*standard deviation of cell control (σc) plus 3*standard deviation of the virus control (σv)/[mean cell control signal (νc) minus mean virus control signal (νv). Zhang, J. H., T. D. Chung, and K. R. Oldenburg, A Simple Statistical Parameter for Use in Evaluation and Validation of High Throughput Screening Assays. J Biomol Screen, 1999. 4(2): p. 67-73.

The signal/background (S/B) was calculated from mean cell control signal (ν_{c}) divided by the mean virus control signal (νᵥ). The signal/noise (S/N) was calculated from mean cell control signal (νc) minus mean virus control signal (νᵥ) divided by the (standard deviation of the cell control signal (ν_{c})² minus the standard deviation of the virus control signal (νᵥ)^{1/2[}2]. Zhang, J. H., T. D. Chung, and K. R. Oldenburg, A Simple Statistical Parameter for Use in Evaluation and Validation of High Throughput Screening Assays. J Biomol Screen, 1999. 4(2): p. 67-73.

An EC₅₀ (for CPE inhibition) and IC₅₀ (for cell viability) were calculated for each substance using the 4 parameter Levenburg-Marquardt algorithm with parameter A locked at 0 and parameter B locked at 100. Standard deviation, normalized chi2 and hill slope were used to evaluate the curves. Values were not extrapolated beyond the tested range of concentrations.

The criteria for determining compound activity are based on percent inhibition of CPE. Of the substances demonstrating activity, they have been scored based on their selectivity index, which is defined as IC₅₀/EC₅₀.

In order to demonstrate the reliability of the assay of the present disclosure, the present assay was compared to the diluted virus RSV assays as discussed herein below.

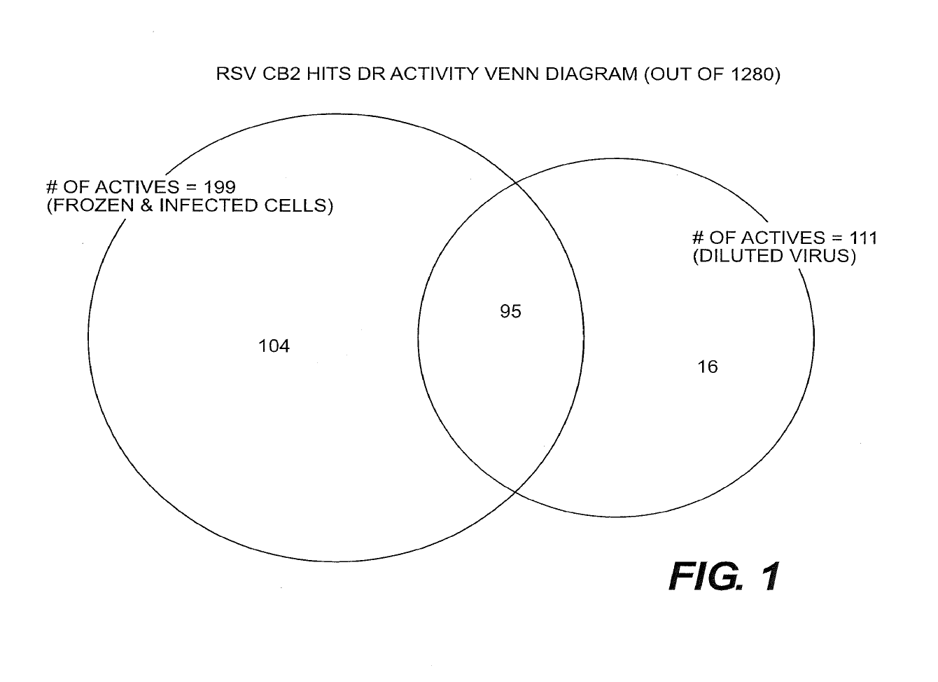

1280 hits selected and cherry-picked from CB2 library were tested in dose response in two RSV assays. One assay used frozen and infected cells according to the present disclosure, the other used diluted virus. EC₅₀ and EC₉₀ results for each compound were calculated. To compare these two assays, EC₅₀ and EC₉₀ results from two parallel assays are examined. The Venn diagram of activities of these compounds from two assays is shown in FIG. 1.

As to the analysis of EC₅₀, only compounds that are active in both assays, and with numerical EC₅₀ values are examined, which makes 92 compounds included in the analysis. For analysis of EC₉₀, 95 compounds that are active in both assays are included in the analysis. However, due to limited number (8) of compounds with both numerical results of EC₉₀, which is not a large enough sample size to make any convincing statistical conclusion, non-numerical EC₉₀ results of these 95 compounds are approximated as below in Table 1:

TABLE 1
Number of compounds with EC₉₀ value approximation (out of 95)

| | Frozen & Infected | Diluted Virus |
|---|---|---|
| | Cells EC₉₀ | EC₉₀ |
| “>1.563” to 1.563 | 1 | 1 |
| “>3.125” to 3.125 | 2 | 2 |
| “>6.25” to 6.25 | 4 | 9 |
| “>12.5” to 12.5 | 9 | 12 |
| “>25” to 25 | 59 | 63 |

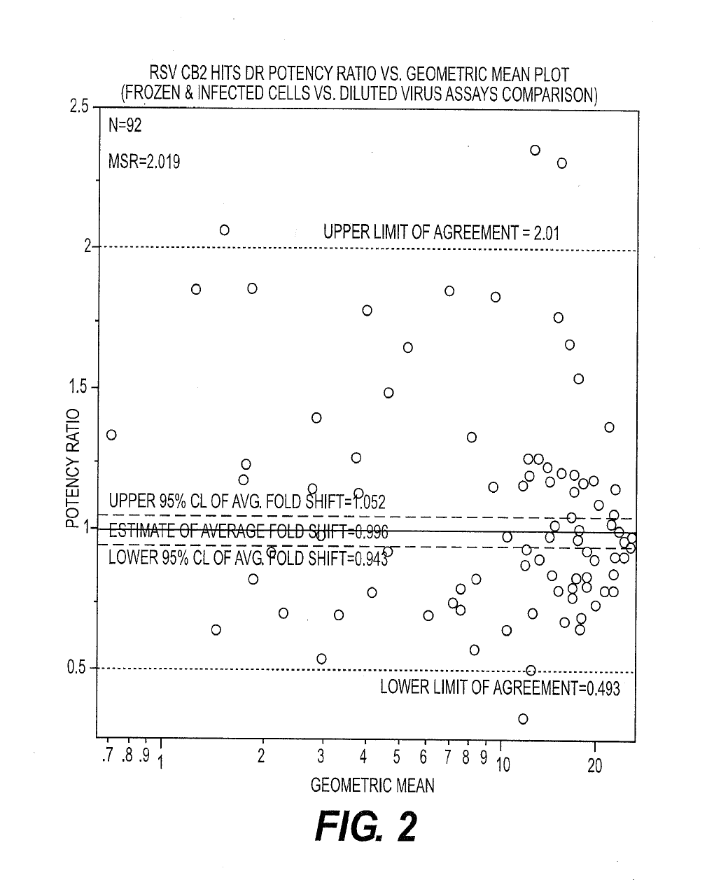

FIG. 2 shows the EC₅₀ analysis, which is a MSR plot of potency ratio vs. geometric means. The Potency Ratio is the EC₅₀ from assay with frozen and infected cells divided by EC₅₀ from diluted cells assay. The horizontal axis is the geometric mean of the two EC₅₀ results of each and every compound. The Minimum Significant Ratio (MSR), Limits of Agreement, and 95% confidence interval of average potency ratio (fold shift) are used to test the quality.

The MSR is the smallest potency ratio between two compounds that is statistically significant. The acceptable values of MSR for a good reproducibility is MSR<3. From this experiment, MSR=2.019, which is within acceptable range, or these two assays are leading to equivalent EC₅₀ results, with 95% confidence, within the interested potency range from 0.049 to 25 pg/ml.

The acceptance range for Limits of Agreement is between 0.33 and 3. From the analysis above, the Limits of Agreement of the two assays is (0.493, 2.01), where both upper and lower limit meet the criteria.

The point estimate of the average fold shift in EC₅₀ is 0.996, which shows, in average, the EC₅₀ result from assay with frozen and infected cells is 99.6% of those from the assay with diluted virus, which is very close to a perfect result of 1 from two exactly equivalent assays. According to the 95% confidence interval of the average potency ratio (fold shift in EC₅₀), which is (0.943, 1.052), 1 is included in this interval. This confirms the conclusion drawn based on the point estimate of potency ratio.

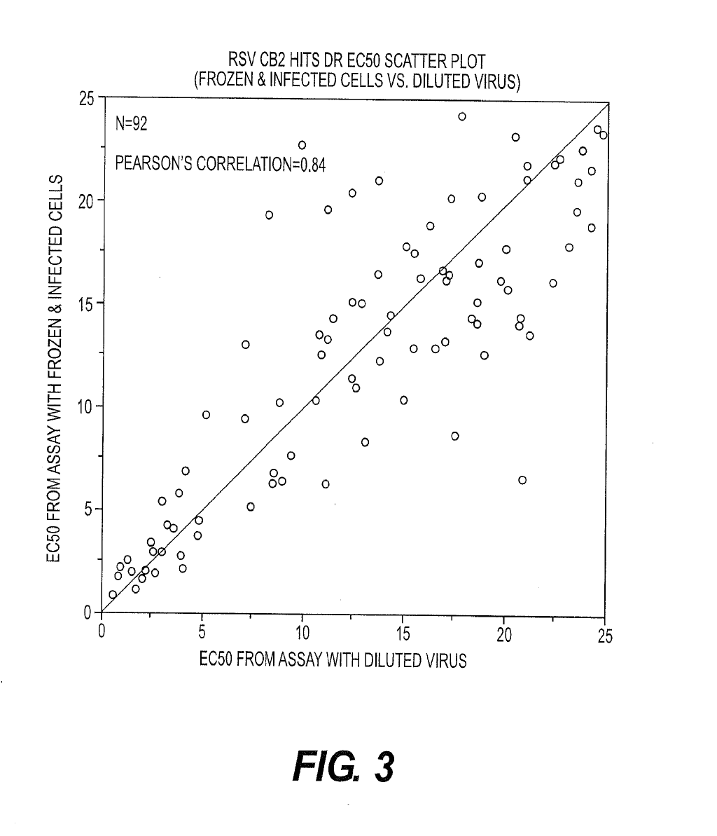

From the scatter plot and analysis in FIG. 3, the correlation of EC₅₀ results from frozen & infected cells vs. diluted virus assays is 0.84, which shows strong correlation and significant linearity.

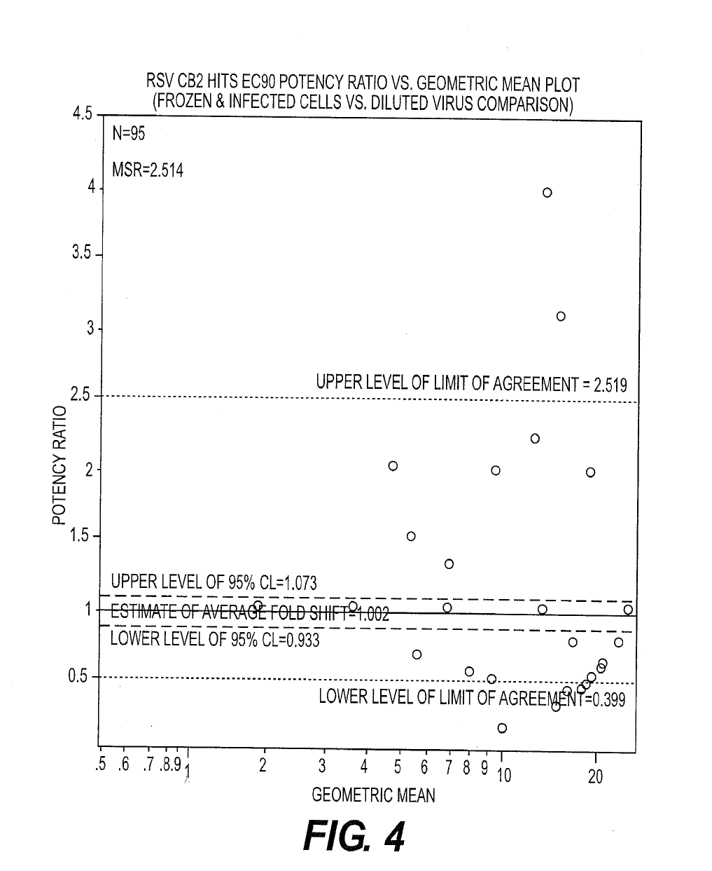

FIG. 4 shows the EC₉₀ analysis, which is a MSR plot of potency ratio vs. geometric means. Potency Ratio is the EC₉₀ from assay with frozen and infected cells divided by EC₉₀ from diluted cells assay. Horizontal axis is the geometric mean of the two EC₉₀ results of every compound. Minimum Significant Ratio (MSR), Limits of Agreement, and 95% confidence interval of average potency ratio (fold shift) are used to test the quality.

From this experiment, MSR=2.514, which is within acceptable range (<3), or these two assays are leading to equivalent EC₉₀ results, with 95% confidence, within the interested potency range from 0.049 to 25 μg/ml.

The acceptance range for Limits of Agreement is between 0.33 and 3. From the analysis above, the Limits of Agreement of the two assays is (0.399, 2.519), where both upper and lower limit meet the criteria.

The point estimate of the average fold shift in EC₉₀ is 1.002, which shows, in average, the EC₉₀ result from assay with the frozen and infected cells is 1.002 times of those from the assay with diluted virus, which is close to an exact result of 1 from two equivalent assays. According to the 95% confidence interval of the average potency ratio (fold shift in EC₉₀), which is (0.933, 1.073), 1 is included in this interval. This confirms the conclusion drawn based on the point estimate of potency ratio.

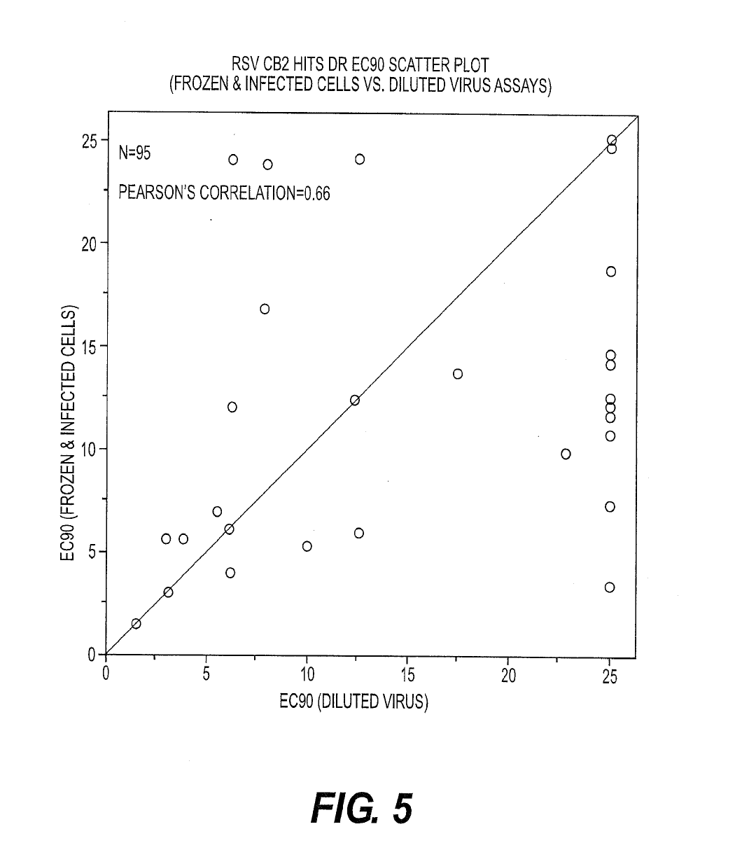

The correlation and scatter plot is shown in FIG. 5. The correlation of EC₉₀ results from frozen & infected cells vs. diluted virus assays is 0.66, which shows moderate correlation and linearity.

The compounds presented below in Table 2 have been identified by the assay of the present disclosure.

TABLE 2

| Compound | | | | | |
|---|---|---|---|---|---|
| ID | Supplier | Supplier ID | Structure | EC₅₀ | CC₅₀ |
| AB00053361 | SRI Repository | SRI-16259 | 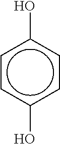 | 47.90 | >50.00 |
| AB00076694 | Chembridge 2 | 5249955 | 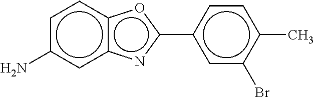 | 16.36 | >25.00 |
| AB00132114 | Chembridge 2 | 7941435 | 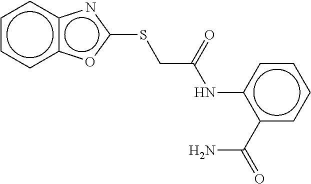 | 20.72 | >25.00 |
| AB00139215 | Chembridge 2 | 5983631 | 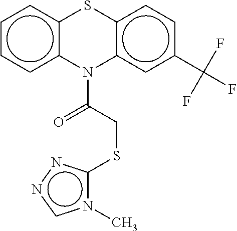 | 10.95 | >25.00 |
| AB00173190 | SRI Repository | SRI-11111 | 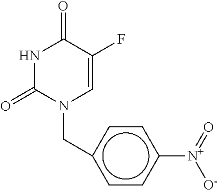 | 20.97 | >50.00 |
| AB00174524 | SRI Repository | SRI-10013 | 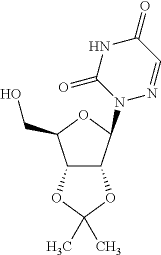 [SMILES: CC1(C)O[C@@H]2[C@H](O1)[C@@H](CO)O[C@H]2n1ncc(=O)[nH]c1=O] | 3.25 | >50.00 |
| AB00275199 | Chembridge 2 | 5128772 | 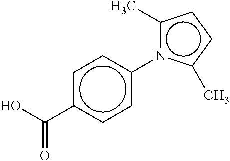 | 13.02 | >25.00 |
| AB00275543 | Chembridge 2 | 5157225 | 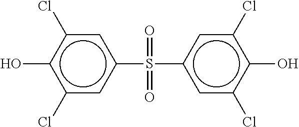 | 16.21 | >25.00 |
| AB00275559 | Chembridge 2 | 5161544 | 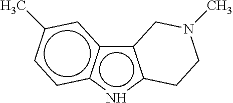 | 6.29 | >25.00 |
| AB00275625 | Chembridge 2 | 5175085 | 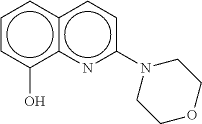 | 15.23 | >25.00 |
| AB00275773 | Chembridge 2 | 5210996 | 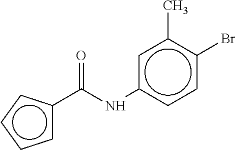 | 10.17 | >25.00 |
| AB00275918 | Chembridge 2 | 5222210 | 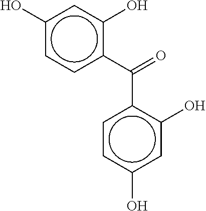 | 20.46 | >25.00 |
| AB00276073 | Chembridge 2 | 5231477 | 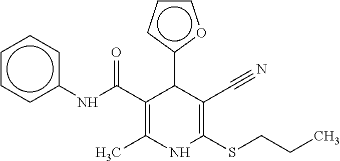 | 5.12 | >25.00 |
| AB00276221 | Chembridge 2 | 5245877 | 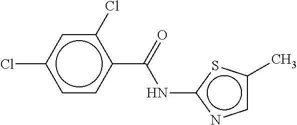 | 17.59 | >25.00 |
| AB00276381 | Chembridge 2 | 5254043 | 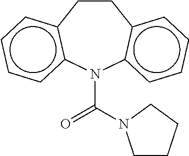 | 12.20 | >25.00 |
| AB00276394 | Chembridge 2 | 5255764 | 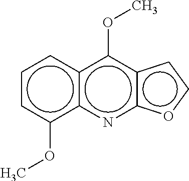 | 15.20 | >25.00 |
| AB00276634 | Chembridge 2 | 5286187 | 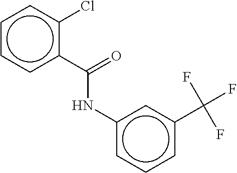 | 15.78 | >25.00 |
| AB00276706 | Chembridge 2 | 5312109 | 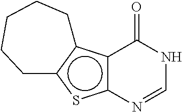 | 19.61 | >25.00 |
| AB00278348 | Chembridge 2 | 5567669 | 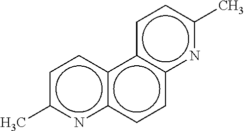 | 12.60 | >25.00 |
| AB00279132 | Chembridge 2 | 5665705 | 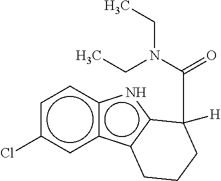 | 1.92 | >25.00 |
| AB00279343 | Chembridge 2 | 5687656 | 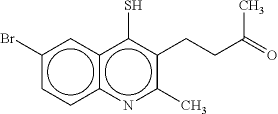 | 20.69 | >25.00 |
| AB00279389 | Chembridge 2 | 5689580 | 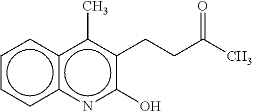 | 14.18 | >25.00 |
| AB00280085 | Chembridge 2 | 5764853 | 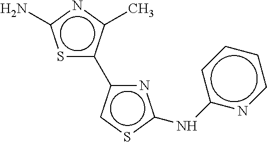 | 18.89 | >25.00 |
| AB00280244 | Chembridge 2 | 5790700 | 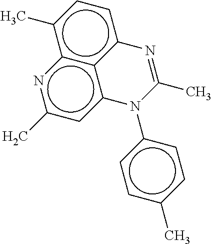 | 8.62 | >25.00 |
| AB00280499 | Chembridge 2 | 5802791 | 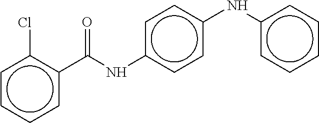 | 6.60 | >25.00 |
| AB00282102 | Chembridge 2 | 5929400 | 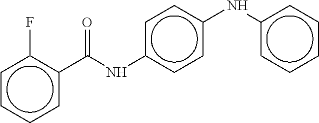 | 11.98 | >25.00 |
| AB00282821 | Chembridge 2 | 6010355 | 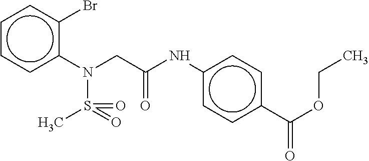 | 14.49 | >25.00 |
| AB00283422 | Chembridge 2 | 6045122 | 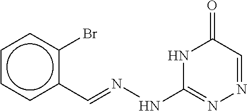 | 15.83 | >25.00 |
| AB00283699 | Chembridge 2 | 6048378 | 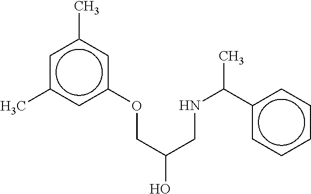 | 12.88 | >25.00 |
| AB00284323 | Chembridge 2 | 6076447 | 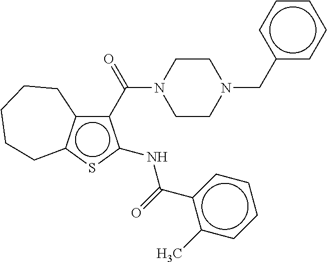 | 17.54 | >25.00 |
| AB00284847 | Chembridge 2 | 6125986 | 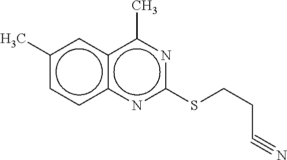 | 22.48 | >25.00 |
| AB00285095 | Chembridge 2 | 6133629 | 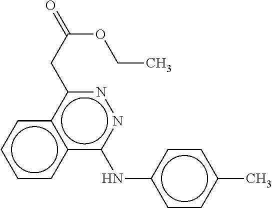 | 9.72 | >25.00 |
| AB00287440 | Chembridge 2 | 6314397 | 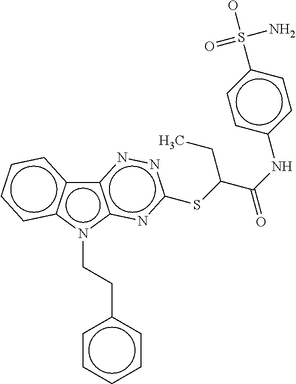 | 17.84 | >25.00 |
| AB00287600 | Chembridge 2 | 6334319 | 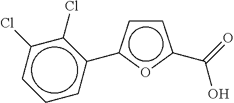 | 12.65 | >25.00 |
| AB00287736 | Chembridge 2 | 6349777 | 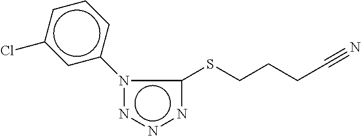 | 16.16 | >25.00 |
| AB00288908 | Chembridge 2 | 6431174 | 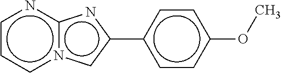 | 21.57 | >25.00 |
| AB00289145 | Chembridge 2 | 6438978 | 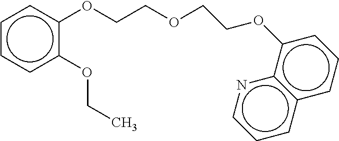 | 24.24 | >25.00 |
| AB00289457 | Chembridge 2 | 6464487 | 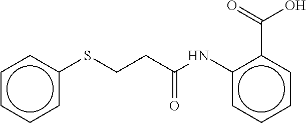 | 2.97 | >25.00 |
| AB00290004 | Chembridge 2 | 6520943 | 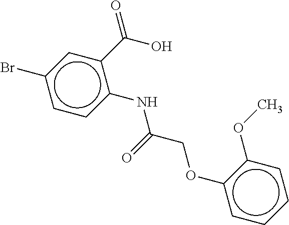 | 23.21 | >25.00 |
| AB00290142 | Chembridge 2 | 6535527 | 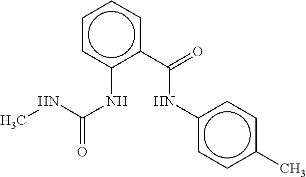 | 19.44 | >25.00 |
| AB00291074 | Chembridge 2 | 6606287 | 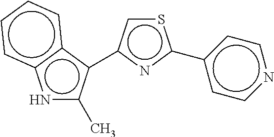 | 20.59 | >25.00 |
| AB00291408 | Chembridge 2 | 6624234 | 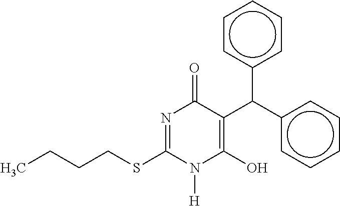 | 24.18 | >25.00 |
| AB00291789 | Chembridge 2 | 6631212 | 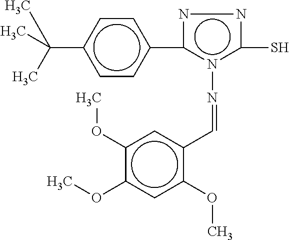 | 10.96 | >25.00 |
| AB00292155 | Chembridge 2 | 6651233 | 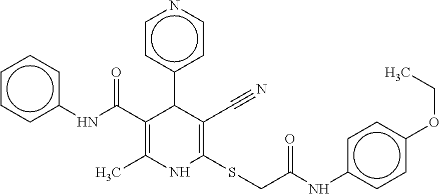 | 15.74 | >25.00 |
| AB00292171 | Chembridge 2 | 6652641 | 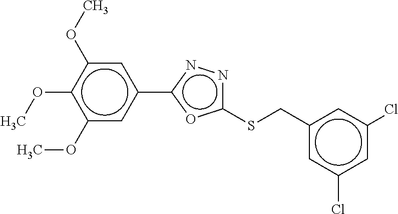 | 7.64 | >25.00 |
| AB00292352 | Chembridge 2 | 6663053 | 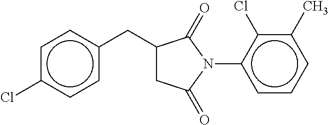 | 8.56 | >25.00 |
| AB00292655 | Chembridge 2 | 6694803 | 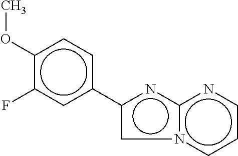 | 10.88 | >25.00 |
| AB00293237 | Chembridge 2 | 6743233 | 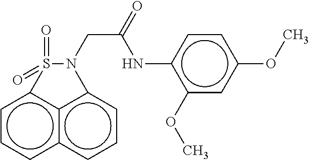 | 10.32 | >25.00 |
| AB00293238 | Chembridge 2 | 6743321 | 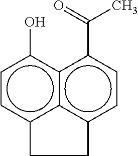 | 11.40 | >25.00 |
| AB00293678 | Chembridge 2 | 6786686 | 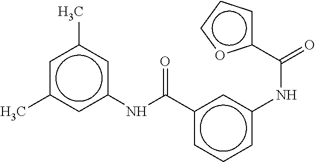 | 18.64 | >25.00 |
| AB00293683 | Chembridge 2 | 6787055 | 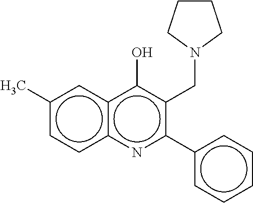 | 18.82 | >25.00 |
| AB00293926 | Chembridge 2 | 6826767 | 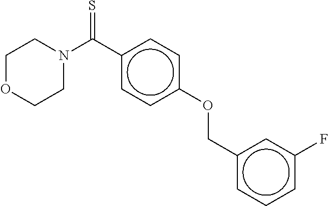 | 10.80 | >25.00 |
| AB00294368 | Chembridge 2 | 6880473 | 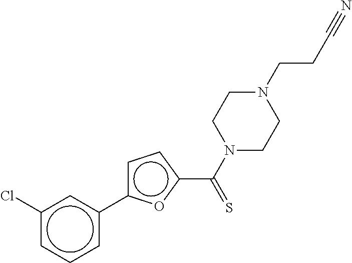 | 14.97 | >25.00 |
| AB00294484 | Chembridge 2 | 6884133 | 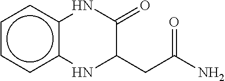 | 16.44 | >25.00 |
| AB00294809 | Chembridge 2 | 6894993 | 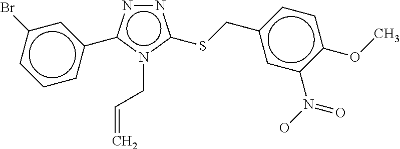 | 20.29 | >25.00 |
| AB00294826 | Chembridge 2 | 6895791 | 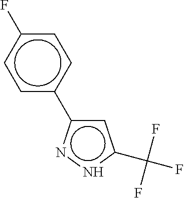 | 16.73 | >25.00 |
| AB00294890 | Chembridge 2 | 6898236 | 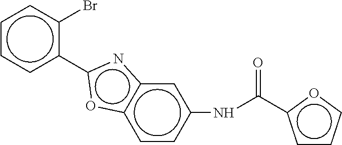 | 22.51 | >25.00 |
| AB00296051 | Chembridge 2 | 6941649 | 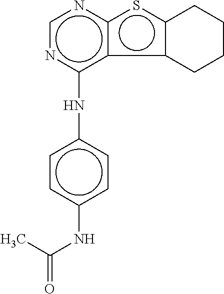 | 16.70 | >25.00 |
| AB00296320 | Chembridge 2 | 6944380 | 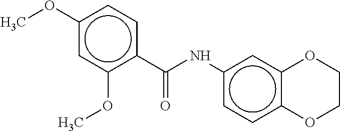 | 8.34 | >25.00 |
| AB00296345 | Chembridge 2 | 6944510 | 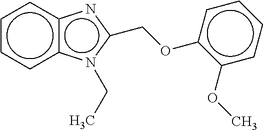 | 18.87 | >25.00 |
| AB00296415 | Chembridge 2 | 6944917 | 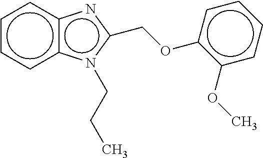 | 4.88 | >25.00 |
| AB00296680 | Chembridge 2 | 6947330 | 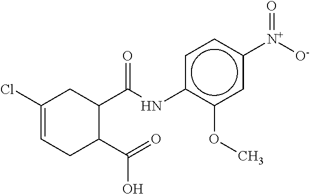 | 12.88 | >25.00 |
| AB00297372 | Chembridge 2 | 6958221 | 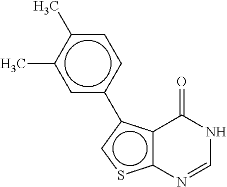 | 10.88 | >25.00 |
| AB00297463 | Chembridge 2 | 6959378 | 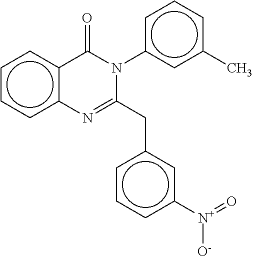 | 16.33 | >25.00 |
| AB00297482 | Chembridge 2 | 6959557 | 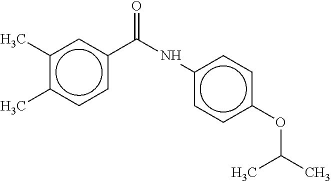 | 14.09 | >25.00 |
| AB00297971 | Chembridge 2 | 6968195 | 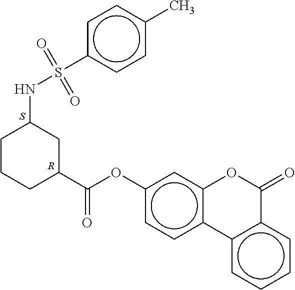 | 19.21 | >25.00 |
| AB00297982 | Chembridge 2 | 6968340 | 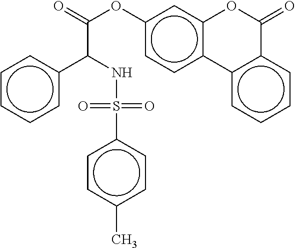 | 11.04 | >25.00 |
| AB00298501 | Chembridge 2 | 6980267 | 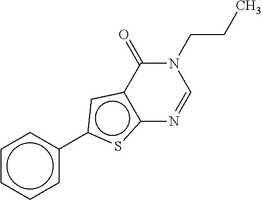 | 3.14 | >25.00 |
| AB00298660 | Chembridge 2 | 6982394 | 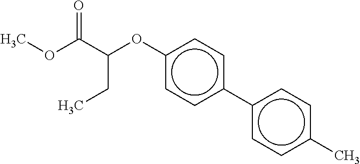 | 13.48 | >25.00 |
| AB00298665 | Chembridge 2 | 6982423 | 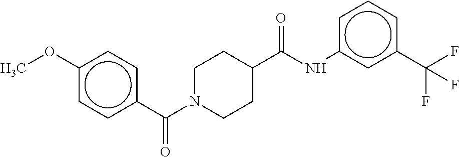 | 12.14 | >25.00 |
| AB00298862 | Chembridge 2 | 6985389 | 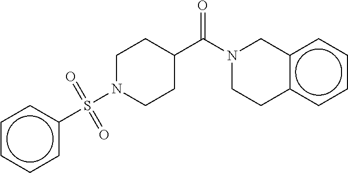 | 20.44 | >25.00 |
| AB00299031 | Chembridge 2 | 6988000 | 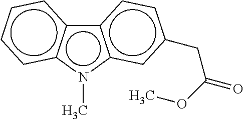 | 21.69 | >25.00 |
| AB00299290 | Chembridge 2 | 6991621 | 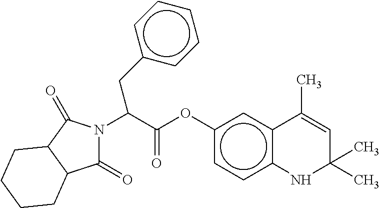 | 15.84 | >25.00 |
| AB00299380 | Chembridge 2 | 6992900 | 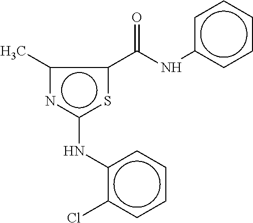 | 10.41 | >25.00 |
| AB00299386 | Chembridge 2 | 6992994 | 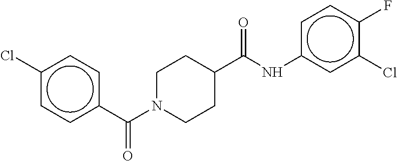 | 12.34 | >25.00 |
| AB00299547 | Chembridge 2 | 6996310 | 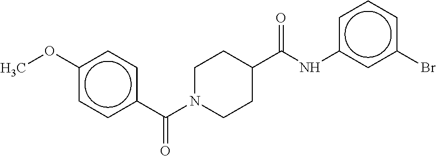 | 18.06 | >25.00 |
| AB00299924 | Chembridge 2 | 7002398 | 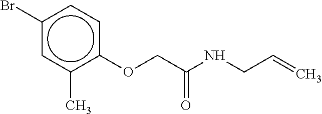 | 22.42 | >25.00 |
| AB00300194 | Chembridge 2 | 7004218 | 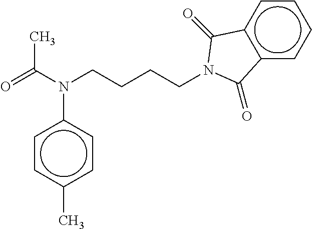 | 9.01 | >25.00 |
| AB00301183 | Chembridge 2 | 7012535 | 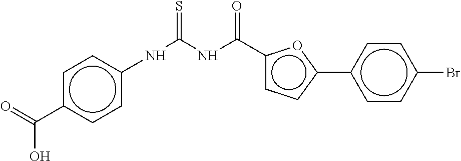 | 22.65 | >25.00 |
| AB00301184 | Chembridge 2 | 7012545 | 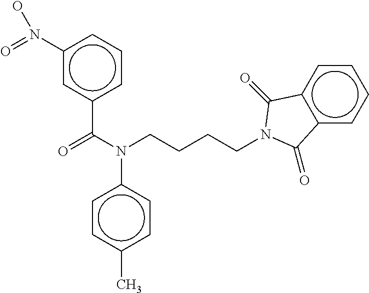 | 2.53 | >25.00 |
| AB00301352 | Chembridge 2 | 7013771 | 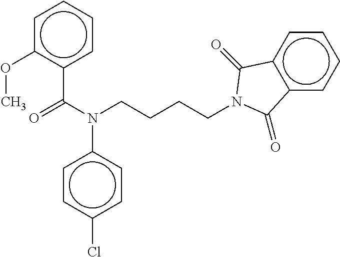 | 21.07 | >25.00 |
| AB00301378 | Chembridge 2 | 7013938 | 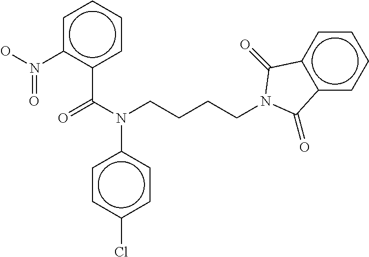 | 17.77 | >25.00 |
| AB00301782 | Chembridge 2 | 7017760 | 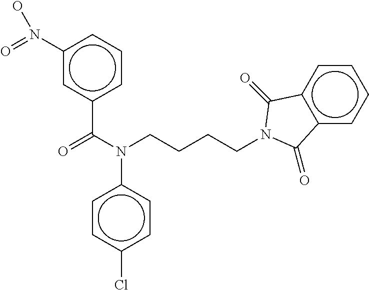 | 2.01 | >25.00 |
| AB00302018 | Chembridge 2 | 7020237 | 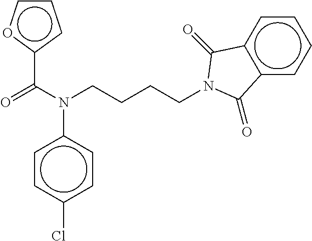 | 3.40 | >25.00 |
| AB00302020 | Chembridge 2 | 7020263 | 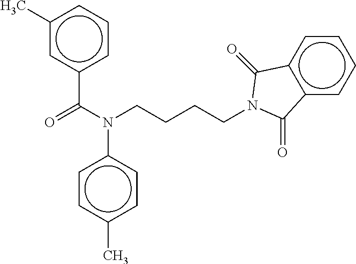 | 15.13 | >25.00 |
| AB00302396 | Chembridge 2 | 7028652 | 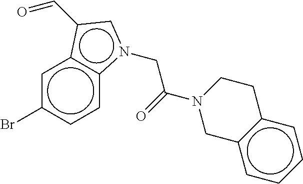 | 23.83 | >25.00 |
| AB00302454 | Chembridge 2 | 7032008 | 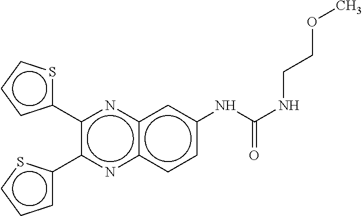 | 15.34 | >25.00 |
| AB00302921 | Chembridge 2 | 7052098 | 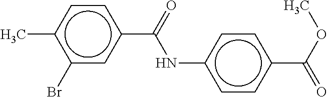 | 20.27 | >25.00 |
| AB00303357 | Chembridge 2 | 7089660 | 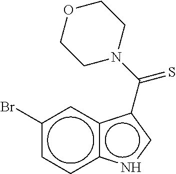 | 21.92 | >25.00 |
| AB00304418 | Chembridge 2 | 7113935 | 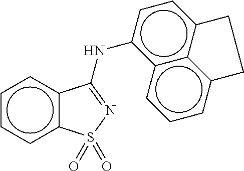 | 10.72 | >25.00 |
| AB00304690 | Chembridge 2 | 7115690 | 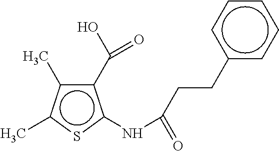 | 13.21 | >25.00 |
| AB00305611 | Chembridge 2 | 7125924 | 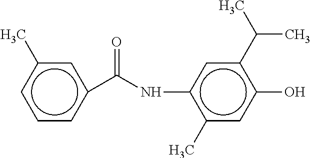 | 17.08 | >25.00 |
| AB00306112 | Chembridge 2 | 7135263 | 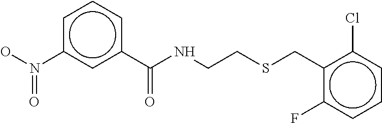 | 5.86 | >25.00 |
| AB00306917 | Chembridge 2 | 7151203 | 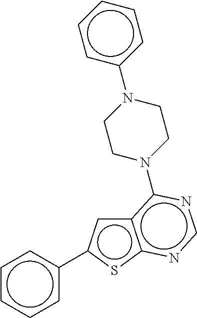 | 17.31 | >25.00 |
| AB00307079 | Chembridge 2 | 7154359 | 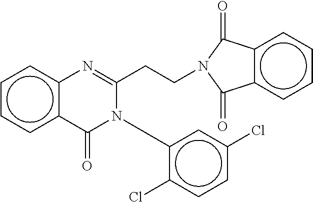 | 14.33 | >25.00 |
| AB00307412 | Chembridge 2 | 7169548 | 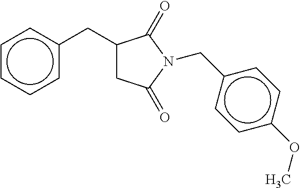 | 21.14 | >25.00 |
| AB00308659 | Chembridge 2 | 7192352 | 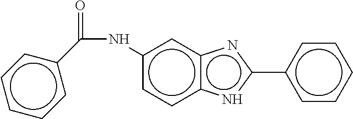 | 8.66 | >25.00 |
| AB00308859 | Chembridge 2 | 7196715 | 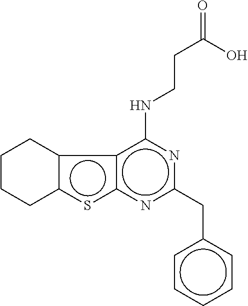 | 20.48 | >25.00 |
| AB00309154 | Chembridge 2 | 7204366 | 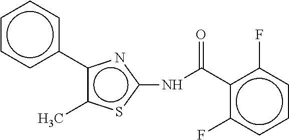 | 6.41 | >25.00 |
| AB00309238 | Chembridge 2 | 7206567 | 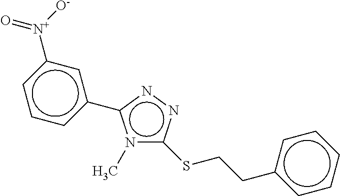 | 20.28 | >25.00 |
| AB00309550 | Chembridge 2 | 7215163 | 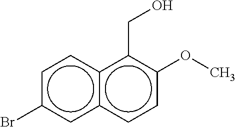 | 16.31 | >25.00 |
| AB00309817 | Chembridge 2 | 7223091 | 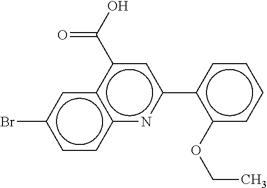 | 14.35 | >25.00 |
| AB00309842 | Chembridge 2 | 7223323 | 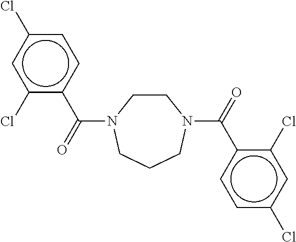 | 12.31 | >25.00 |
| AB00309859 | Chembridge 2 | 7223481 | 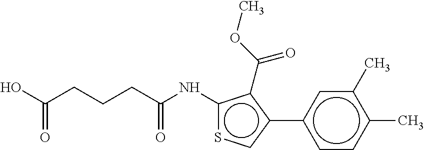 | 6.84 | >25.00 |
| AB00310156 | Chembridge 2 | 7227882 | 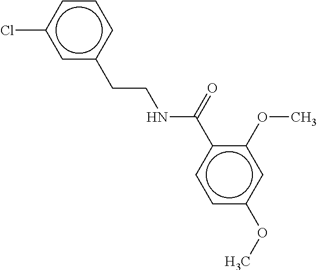 | 10.38 | >25.00 |
| AB00310224 | Chembridge 2 | 7228740 | 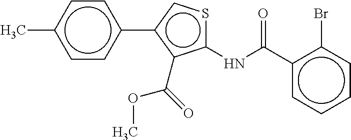 | 24.46 | >25.00 |
| AB00310729 | Chembridge 2 | 7236843 | 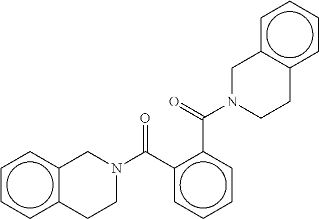 | 14.11 | >25.00 |
| AB00310739 | Chembridge 2 | 7236979 | 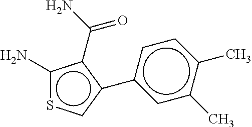 | 6.31 | >25.00 |
| AB00310767 | Chembridge 2 | 7237493 | 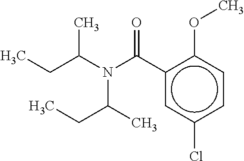 | 15.04 | >25.00 |
| AB00310808 | Chembridge 2 | 7238138 | 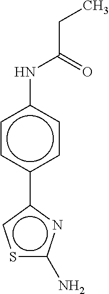 | 23.65 | >25.00 |
| AB00310846 | Chembridge 2 | 7238770 | 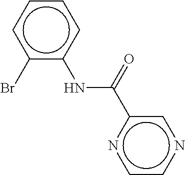 | 19.62 | >25.00 |
| AB00310910 | Chembridge 2 | 7239916 | 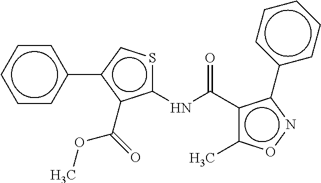 | 1.15 | >25.00 |
| AB00311298 | Chembridge 2 | 7247789 | 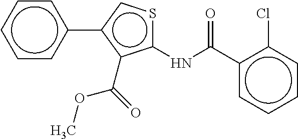 | 7.72 | >25.00 |
| AB00311411 | Chembridge 2 | 7250049 | 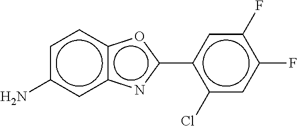 | 22.70 | >25.00 |
| AB00311948 | Chembridge 2 | 7257552 | 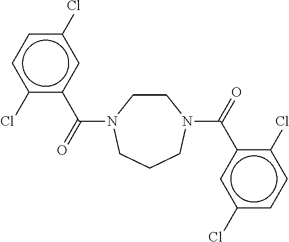 | 2.32 | >25.00 |
| AB00312102 | Chembridge 2 | 7259967 | 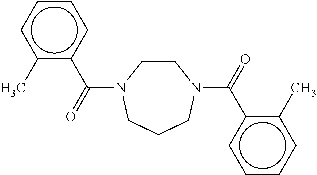 | 3.71 | >25.00 |
| AB00313012 | Chembridge 2 | 7275865 | 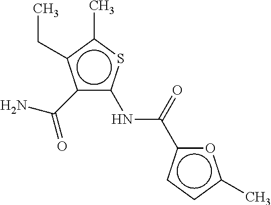 | 17.97 | >25.00 |
| AB00313042 | Chembridge 2 | 7276383 | 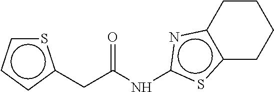 | 1.89 | >25.00 |
| AB00313091 | Chembridge 2 | 7277298 | 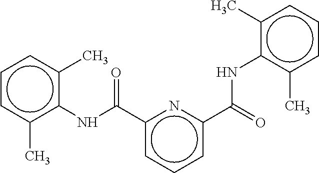 | 19.46 | >25.00 |
| AB00313218 | Chembridge 2 | 7279713 | 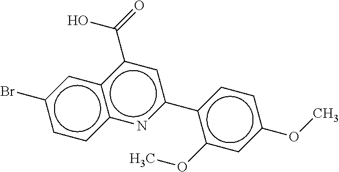 | 22.18 | >25.00 |
| AB00313952 | Chembridge 2 | 7305797 | 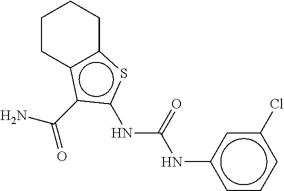 | 2.72 | >25.00 |
| AB00314111 | Chembridge 2 | 7310127 | 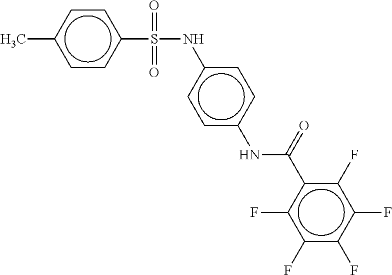 | 6.97 | >25.00 |
| AB00314248 | Chembridge 2 | 7314590 | 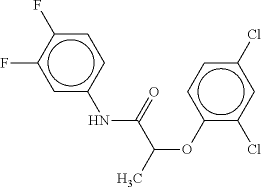 | 23.83 | >25.00 |
| AB00314279 | Chembridge 2 | 7315214 | 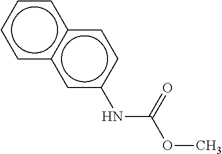 | 23.47 | >25.00 |
| AB00315446 | Chembridge 2 | 7354607 | 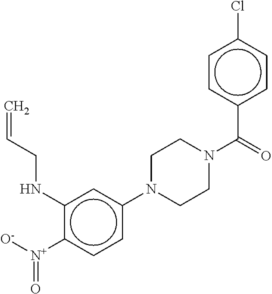 | 12.00 | >25.00 |
| AB00315731 | Chembridge 2 | 7356780 | 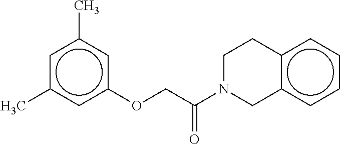 | 21.07 | >25.00 |
| AB00315918 | Chembridge 2 | 7358180 | 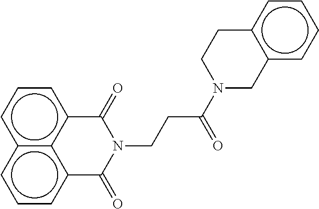 | 15.37 | >25.00 |
| AB00316243 | Chembridge 2 | 7362106 | 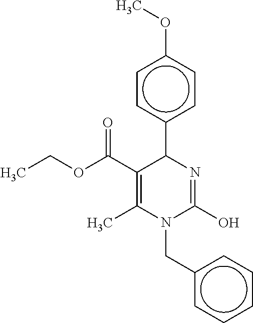 | 18.51 | >25.00 |
| AB00317095 | Chembridge 2 | 7373193 | 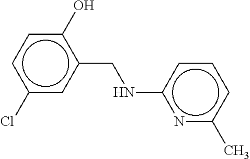 | 24.33 | >25.00 |
| AB00317165 | Chembridge 2 | 7374281 | 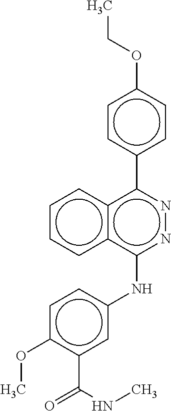 | 19.46 | >25.00 |
| AB00317214 | Chembridge 2 | 7375133 | 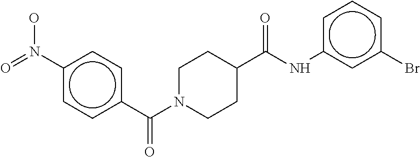 | 12.36 | >25.00 |
| AB00317272 | Chembridge 2 | 7376125 | 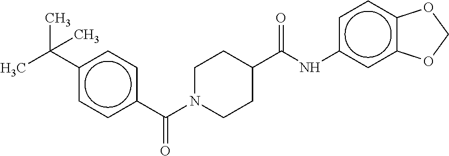 | 22.28 | >25.00 |
| AB00317410 | Chembridge 2 | 7378526 | 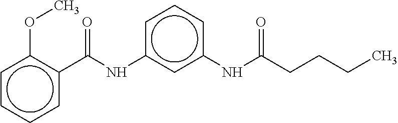 | 22.84 | >25.00 |
| AB00317604 | Chembridge 2 | 7382761 | 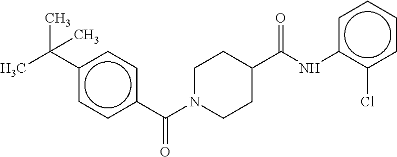 | 23.69 | >25.00 |
| AB00317685 | Chembridge 2 | 7384133 | 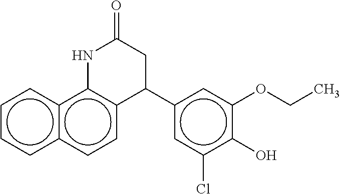 | 20.88 | >25.00 |
| AB00318114 | Chembridge 2 | 7396766 | 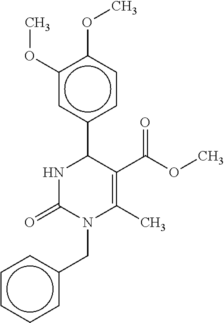 | 3.37 | >25.00 |
| AB00318264 | Chembridge 2 | 7402594 | 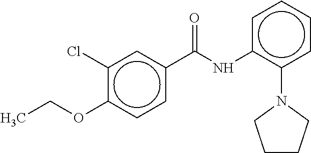 | 22.89 | >25.00 |
| AB00318335 | Chembridge 2 | 7403644 | 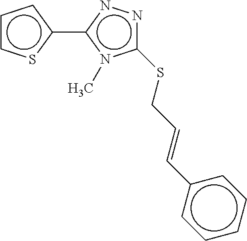 | 24.18 | >25.00 |
| AB00318401 | Chembridge 2 | 7404485 | 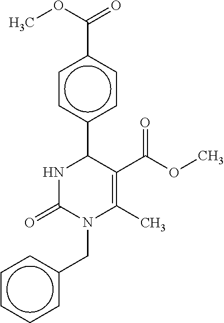 | 2.39 | >25.00 |
| AB00318606 | Chembridge 2 | 7407414 | 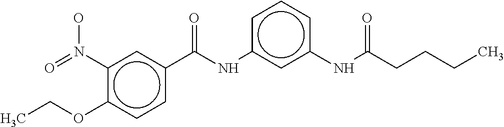 | 23.46 | >25.00 |
| AB00318952 | Chembridge 2 | 7413330 | 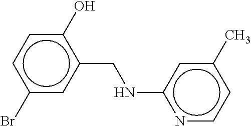 | 22.23 | >25.00 |
| AB00319298 | Chembridge 2 | 7420864 | 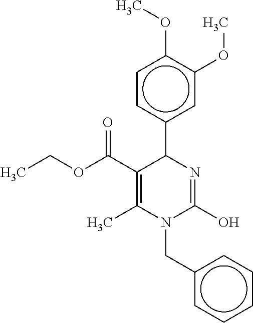 | 2.76 | >25.00 |
| AB00319373 | Chembridge 2 | 7422280 | 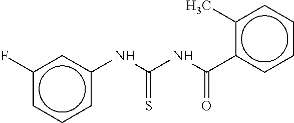 | 24.42 | >25.00 |
| AB00319534 | Chembridge 2 | 7424977 | 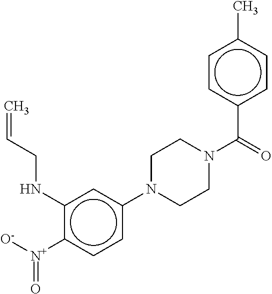 | 13.77 | >25.00 |
| AB00321587 | Chembridge 2 | 7495455 | 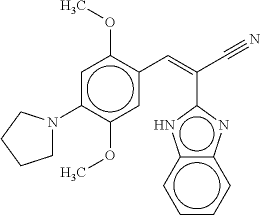 | 9.15 | >25.00 |
| AB00322577 | Chembridge 2 | 7510193 | 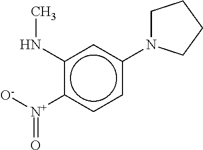 | 18.67 | >25.00 |
| AB00326733 | Chembridge 2 | 7581498 | 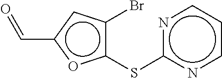 | 18.75 | >25.00 |
| AB00335740 | Chembridge 2 | 7680889 | 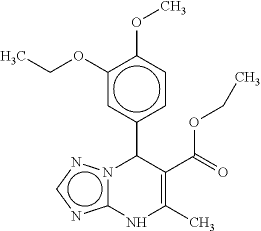 | 21.07 | >25.00 |
| AB00339206 | Chembridge 2 | 7721729 | 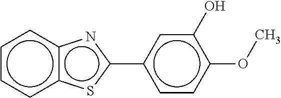 | 21.87 | >25.00 |
| AB00339302 | Chembridge 2 | 7722014 | 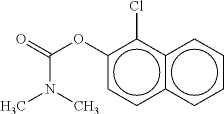 | 12.28 | >25.00 |
| AB00340723 | Chembridge 2 | 7727457 | 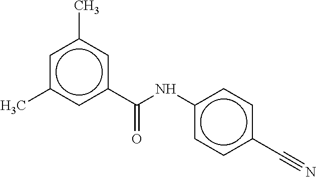 | 19.75 | >25.00 |
| AB00341850 | Chembridge 2 | 7735547 | 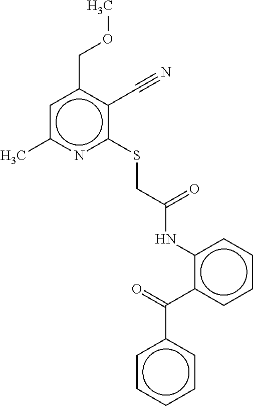 | 2.19 | >25.00 |
| AB00342188 | Chembridge 2 | 7738549 | 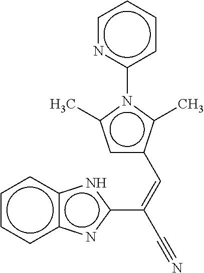 | 13.11 | >25.00 |
| AB00344427 | Chembridge 2 | 7756992 | 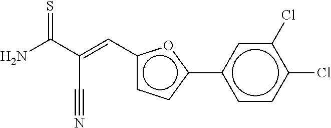 | 17.87 | >25.00 |
| AB00345103 | Chembridge 2 | 7767847 | 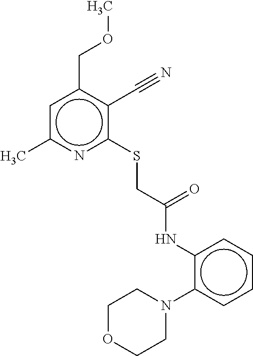 | 2.18 | >25.00 |
| AB00348154 | Chembridge 2 | 7790928 | 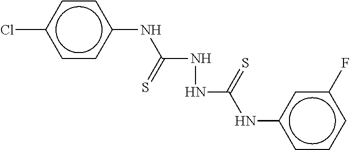 | 10.62 | >25.00 |
| AB00348268 | Chembridge 2 | 7791503 | 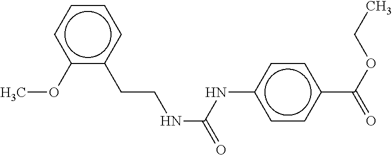 | 21.45 | >25.00 |
| AB00348716 | Chembridge 2 | 7793889 | 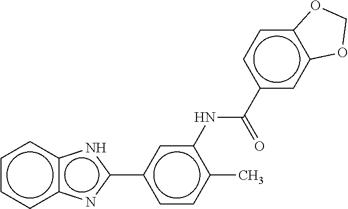 | 17.26 | >25.00 |
| AB00349666 | Chembridge 2 | 7799403 | 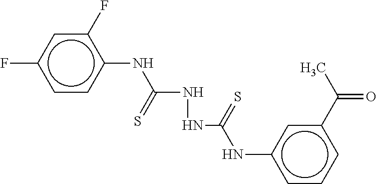 | 21.17 | >25.00 |
| AB00349705 | Chembridge 2 | 7799604 | 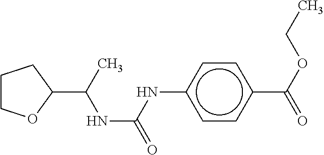 | 19.24 | >25.00 |
| AB00351451 | Chembridge 2 | 7810486 | 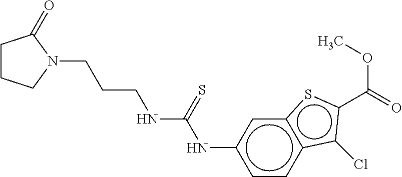 | 17.51 | >25.00 |
| AB00353001 | Chembridge 2 | 7823760 | 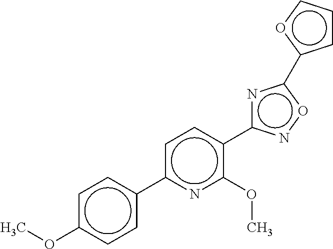 | 18.43 | >25.00 |
| AB00353228 | Chembridge 2 | 7826467 | 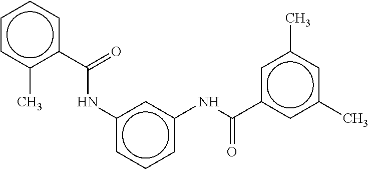 | 9.78 | >25.00 |
| AB00355020 | Chembridge 2 | 7844977 | 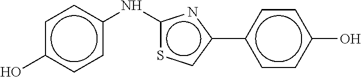 | 2.77 | >25.00 |
| AB00355044 | Chembridge 2 | 7845082 | 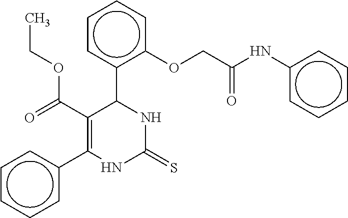 | 6.39 | >25.00 |
| AB00356285 | Chembridge 2 | 7853019 | 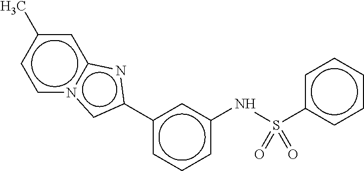 | 4.04 | >25.00 |
| AB00356597 | Chembridge 2 | 7855992 | 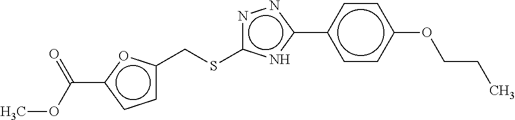 | 11.59 | >25.00 |
| AB00358081 | Chembridge 2 | 7873254 | 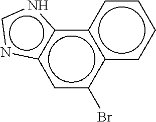 | 12.07 | >25.00 |
| AB00358122 | Chembridge 2 | 7873874 | 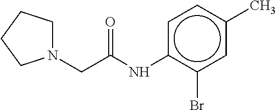 | 20.13 | >25.00 |
| AB00358926 | Chembridge 2 | 7885310 | 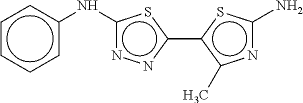 | 12.65 | >25.00 |
| AB00359127 | Chembridge 2 | 7887469 | 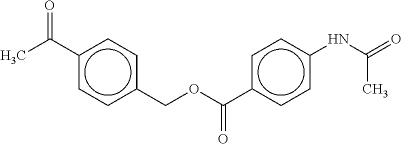 | 18.92 | >25.00 |
| AB00359299 | Chembridge 2 | 7889870 | 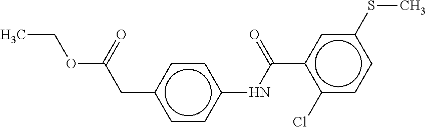 | 6.76 | >25.00 |
| AB00359685 | Chembridge 2 | 7900141 | 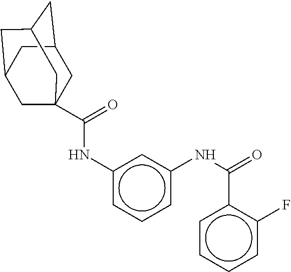 | 9.42 | >25.00 |
| AB00359707 | Chembridge 2 | 7900656 | 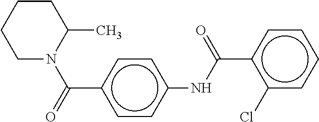 | 11.38 | >25.00 |
| AB00361531 | Chembridge 2 | 7908294 | 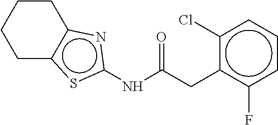 | 0.83 | >25.00 |
| AB00362388 | Chembridge 2 | 7913440 | 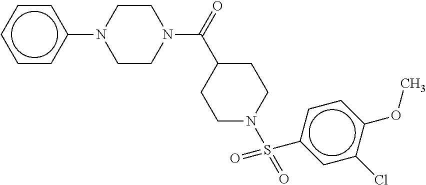 | 16.96 | >25.00 |
| AB00362750 | Chembridge 2 | 7915246 | 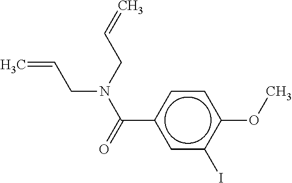 | 21.04 | >25.00 |
| AB00362810 | Chembridge 2 | 7915470 | 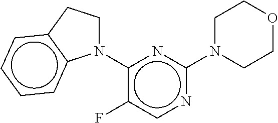 | 21.06 | >25.00 |
| AB00362846 | Chembridge 2 | 7915886 | 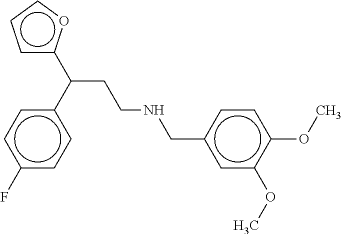 | 4.47 | >25.00 |
| AB00362891 | Chembridge 2 | 7916224 | 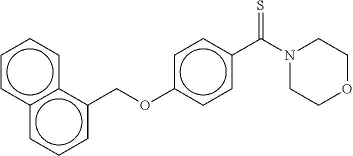 | 23.40 | >25.00 |
| AB00363547 | Chembridge 2 | 7921332 | 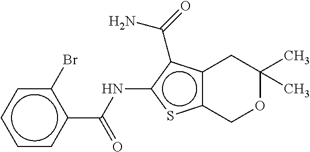 | 13.33 | >25.00 |
| AB00363859 | Chembridge 2 | 7925187 | 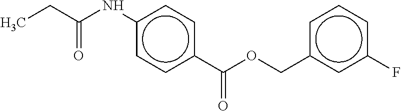 | 12.60 | >25.00 |
| AB00364099 | Chembridge 2 | 7926973 | 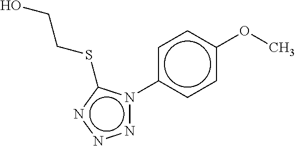 | 19.25 | >25.00 |
| AB00364575 | Chembridge 2 | 7929964 | 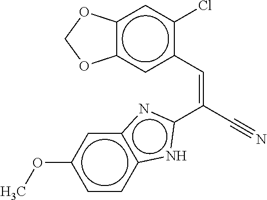 | 17.76 | >25.00 |
| AB00364640 | Chembridge 2 | 7930397 | 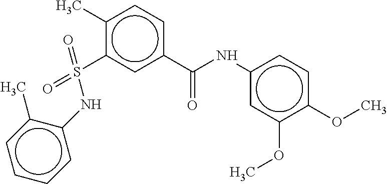 | 11.54 | >25.00 |
| AB00364858 | Chembridge 2 | 7932123 | 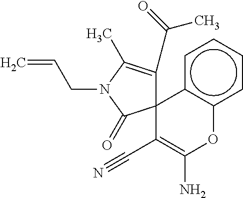 | 21.21 | >25.00 |
| AB00365318 | Chembridge 2 | 7935438 | 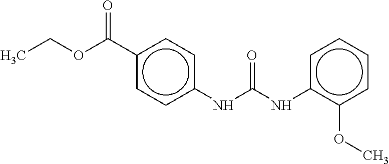 | 11.56 | >25.00 |
| AB00365647 | Chembridge 2 | 7937929 | 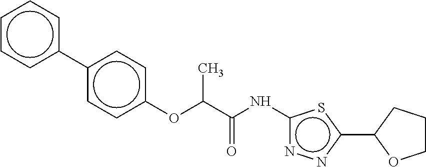 | 19.30 | >25.00 |
| AB00366410 | Chembridge 2 | 7943717 | 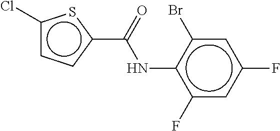 | 20.23 | >25.00 |
| AB00367559 | Chembridge 2 | 7947858 | 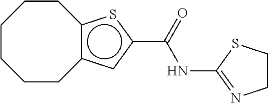 | 19.58 | >25.00 |
| AB00367930 | Chembridge 2 | 7949301 | 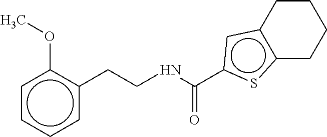 | 5.77 | >25.00 |
| AB00368222 | Chembridge 2 | 7950829 | 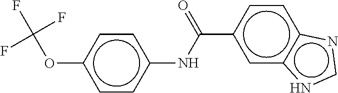 | 12.26 | >25.00 |
| AB00368252 | Chembridge 2 | 7951033 | 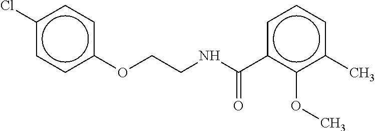 | 9.43 | >25.00 |
| AB00369287 | Chembridge 2 | 7957878 | 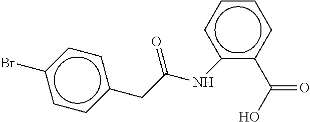 | 9.56 | >25.00 |
| AB00369510 | Chembridge 2 | 7959554 | 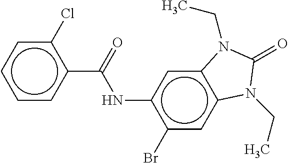 | 10.42 | >25.00 |
| AB00369798 | Chembridge 2 | 7961748 | 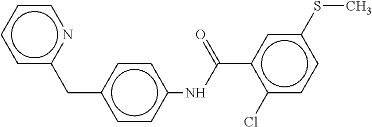 | 11.60 | >25.00 |
| AB00369924 | Chembridge 2 | 7962703 | 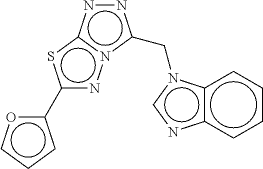 | 1.71 | >25.00 |
| AB00369934 | Chembridge 2 | 7962752 | 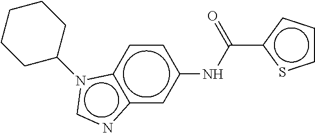 | 13.63 | >25.00 |
| AB00370002 | Chembridge 2 | 7963355 | 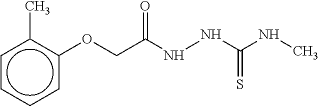 | 22.02 | >25.00 |
| AB00370063 | Chembridge 2 | 7963817 | 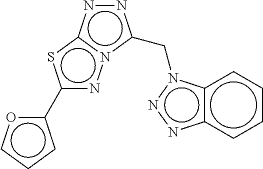 | 1.97 | >25.00 |
| AB00370331 | Chembridge 2 | 7966004 | 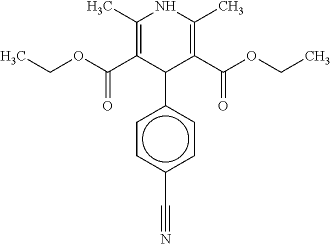 | 4.20 | >25.00 |
| AB00370446 | Chembridge 2 | 7967599 | 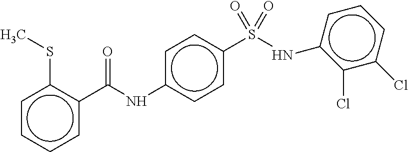 | 4.37 | >25.00 |
| AB00370838 | Chembridge 2 | 7973006 | 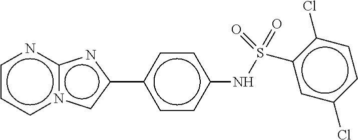 | 22.46 | >25.00 |
| AB00370949 | Chembridge 2 | 7974136 | 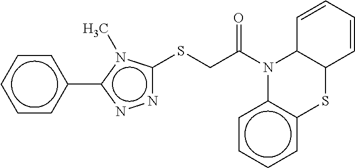 | 18.84 | >25.00 |
| AB00371387 | Chembridge 2 | 7979329 | 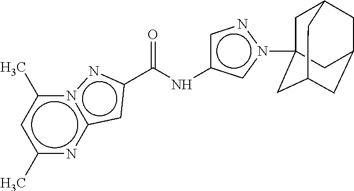 | 12.65 | >25.00 |
| AB00371866 | Chembridge 2 | 7985842 | 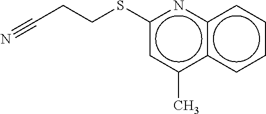 | 13.72 | >25.00 |
| AB00372085 | Chembridge 2 | 7989445 | 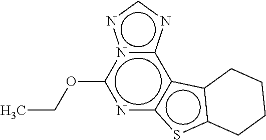 | 11.64 | >25.00 |
| AB00372475 | Chembridge 2 | 7994058 | 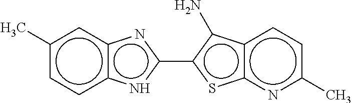 | 21.04 | >25.00 |
| AB00372624 | Chembridge 2 | 7995866 | 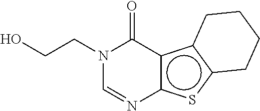 | 23.60 | >25.00 |
| AB00373143 | Chembridge 2 | 9003855 | 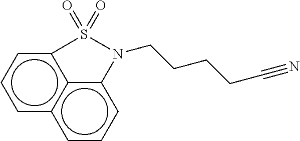 | 14.27 | >25.00 |
| AB00373945 | SRI Repository | SRI-4094 | 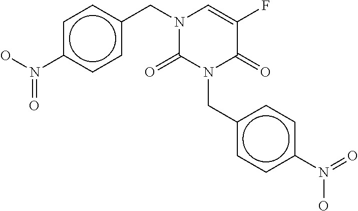 | 13.26 | >50.00 |
| AB00374695 | SRI Repository | SRI-15298 | 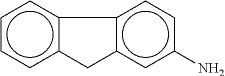 | 46.84 | >50.00 |
| AB00375242 | SRI Repository | SRI-13404 | 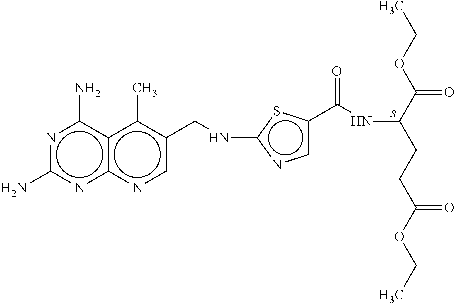 | 41.95 | >50.00 |
| AB00375379 | SRI Repository | SRI-15666 | 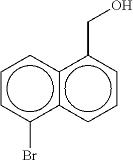 | 39.16 | >50.00 |
| AB00430151 | SRI Repository | SRI-1594 | 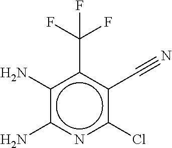 | 38.41 | >50.00 |
| AB00430481 | SRI Repository | SRI-15875 | 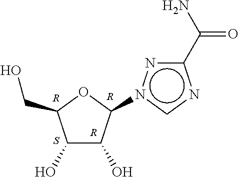 | 38.80 | >50.00 |
| AB00430484 | SRI Repository | SRI-16022 | 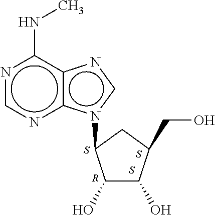 | 33.70 | >50.00 |
| AB00443629 | SRI Repository | SRI-16681 | 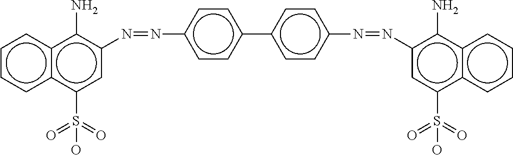 | 35.64 | >50.00 |
| AB00443953 | SRI Repository | SRI-18287 | 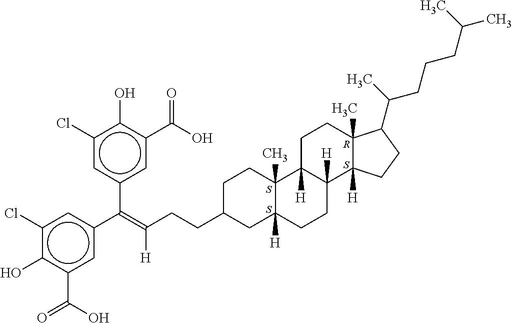 | 21.34 | >50.00 |
| AB00444131 | SRI Repository | SRI-18727 | 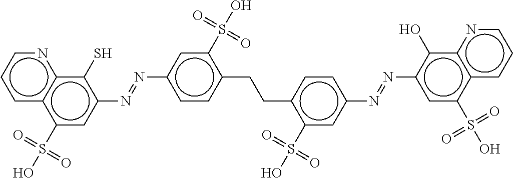 | 18.78 | >50.00 |
| AB00444372 | SRI Repository | SRI-21483 | 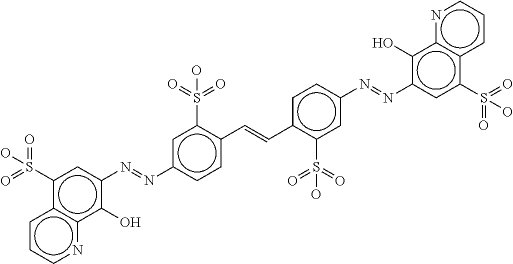 | 10.87 | >50.00 |
| AB00514356 | SRI Repository | SRI-17934 | 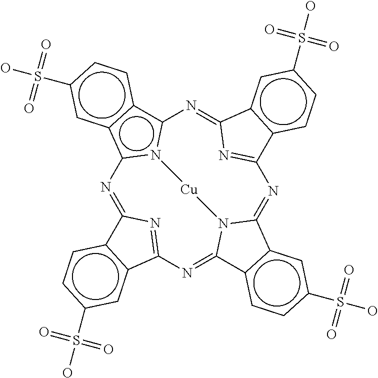 | 23.40 | >50.00 |
| AB00514453 | SRI Repository | SRI-19094 | 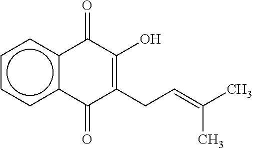 | 7.23 | >50.00 |
| AB00536652 | Enamine | T5242183 | 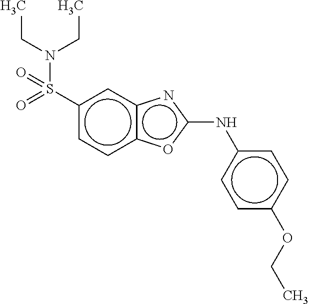 | 38.72 | >50.00 |
| AB00695369 | Profectus | SRI-22810 | 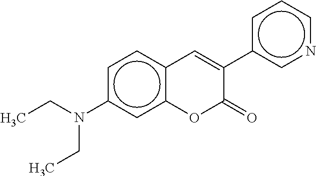 | 35.78 | >50.00 |
| AB00700560 | SRI Repository | SRI-23046 | 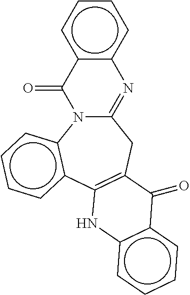 | 11.29 | >50.00 |
| AB00709376 | Enamine | T0501-6404 | 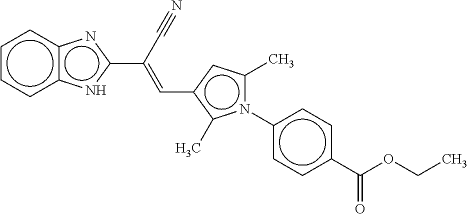 | 10.80 | >50.00 |
| AB00712667 | Enamine | T0508-7698 | 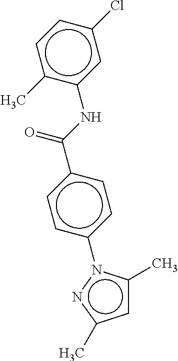 | 23.98 | >50.00 |
| AB00712684 | Enamine | T0508-9789 | 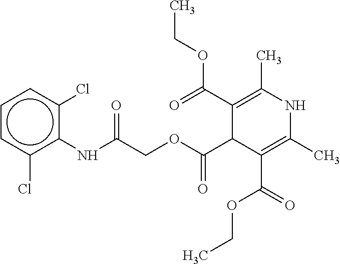 | 21.91 | >50.00 |
| AB00717474 | Enamine | T5499063 | 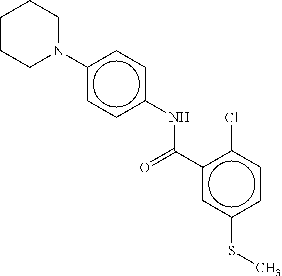 | 12.17 | >50.00 |
| AB00718339 | Enamine | T0516-5070 | 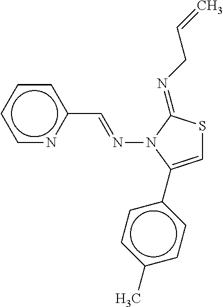 | 1.14 | >50.00 |
| AB00718457 | Enamine | T0516-7402 | 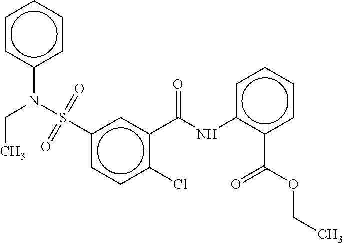 | 23.84 | >50.00 |
| AB00719359 | Enamine | T0516-8387 | 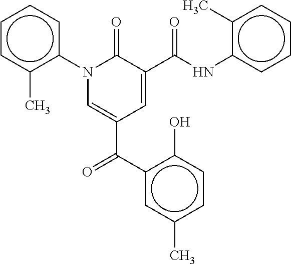 | 29.77 | >50.00 |
| AB00720414 | Enamine | T0518-6946 | 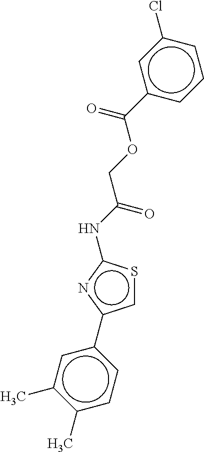 | 49.15 | >50.00 |
| AB00721928 | Enamine | T0520-4430 | 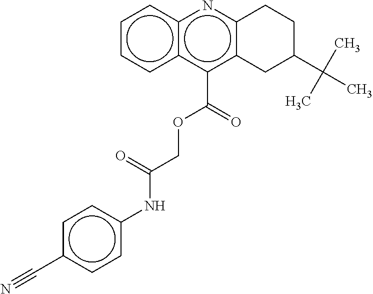 | 41.90 | >50.00 |
| AB00722846 | Enamine | T5221190 | 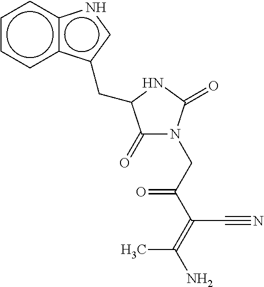 | 1.74 | >50.00 |
| AB00723775 | Enamine | T5234633 | 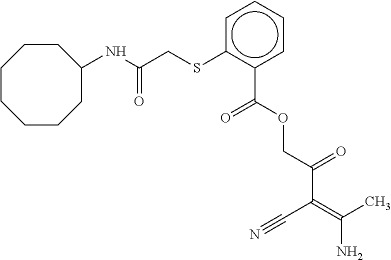 | 23.43 | >50.00 |
| AB00724801 | Enamine | T5248808 | 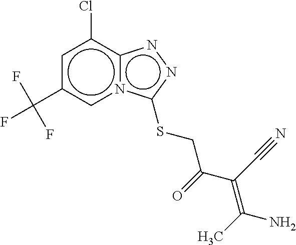 | 16.25 | >50.00 |
| AB00725240 | Enamine | T5255725 | 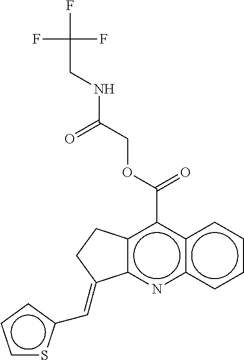 | 43.31 | >50.00 |
| AB00725275 | Enamine | T5256425 | 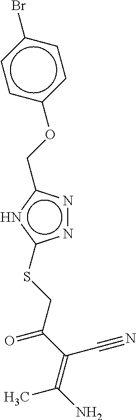 | 28.48 | >50.00 |
| AB00725538 | Enamine | T5259183 | 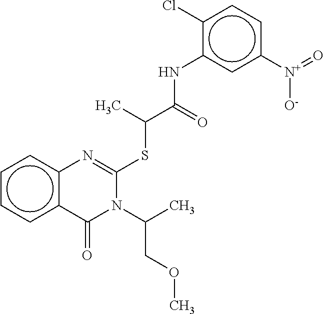 | 6.30 | >50.00 |
| AB00726710 | Enamine | T5269946 | 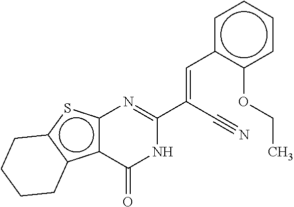 | 30.87 | >50.00 |
| AB00728303 | Enamine | T5286991 | 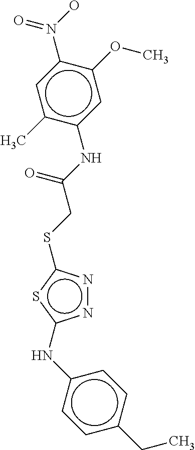 | 28.29 | >50.00 |
| AB00728757 | Enamine | T5293387 | 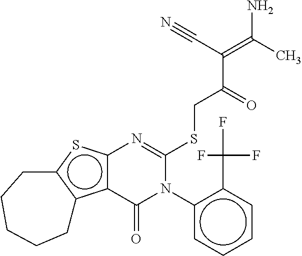 | 18.11 | >50.00 |
| AB00728760 | Enamine | T5293403 | 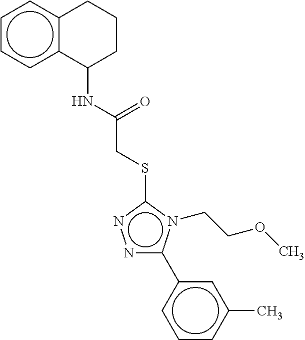 | 37.89 | >50.00 |
| AB00739576 | Enamine | T5497119 | 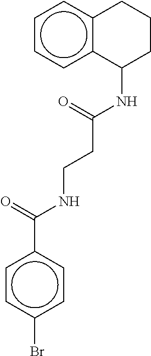 | 24.71 | >50.00 |
| AB00739796 | Enamine | T5531438 | 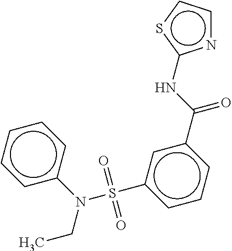 | 22.23 | >50.00 |
| AB00741842 | Enamine | T5493974 | 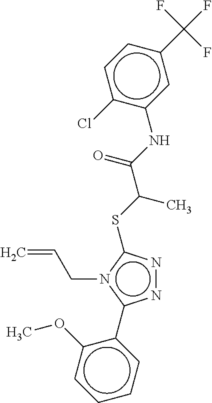 | 26.67 | >50.00 |
| AB00747970 | Enamine | T5535819 | 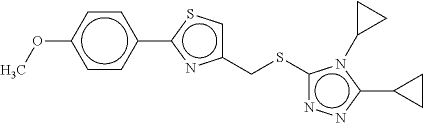 | 10.75 | >50.00 |
| AB00756405 | Enamine | T5647248 | 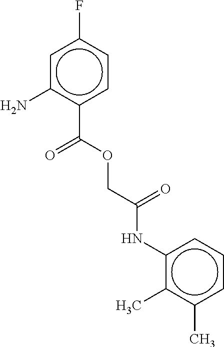 | 11.45 | >50.00 |
| AB00764110 | Enamine | T5729768 | 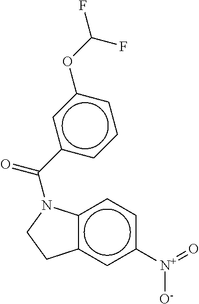 | 41.53 | >50.00 |
| AB00766545 | Enamine | T5770620 | 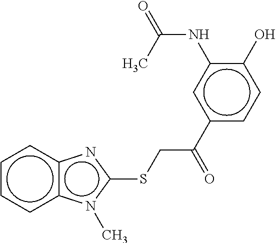 | 26.15 | >50.00 |
| AB00767910 | Enamine | T5788513 | 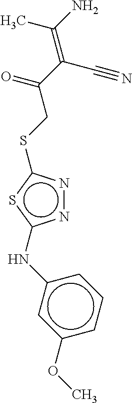 | 6.07 | >50.00 |
| AB00776619 | Enamine | T5872935 | 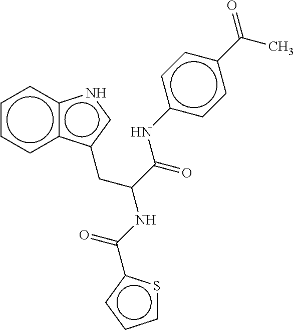 | 27.27 | >50.00 |
| AB00784993 | Enamine | T5948830 | 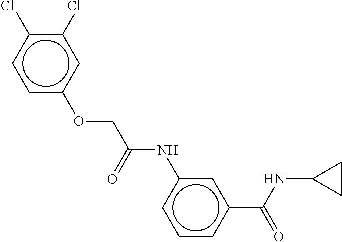 | 12.04 | >50.00 |
| AB00785032 | Enamine | T5949075 | 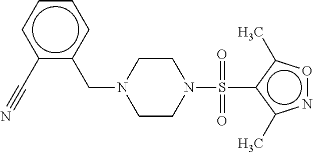 | 46.45 | >50.00 |
| AB00785138 | Enamine | T5949368 | 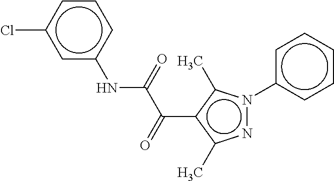 | 7.52 | >50.00 |
| AB00785889 | Enamine | T5941086 | 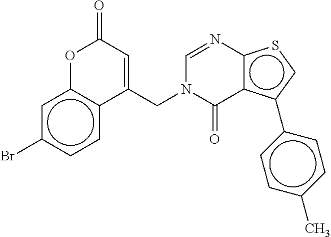 | 23.40 | >50.00 |
| AB00796257 | Enamine | T5829062 | 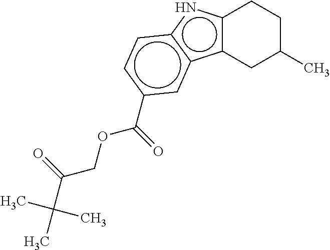 | 24.84 | >50.00 |
| AB00796961 | Enamine | 16006611 | 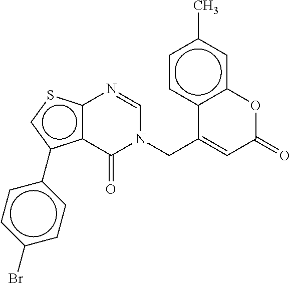 | 31.85 | >50.00 |
| AB00796970 | Enamine | 16006652 | 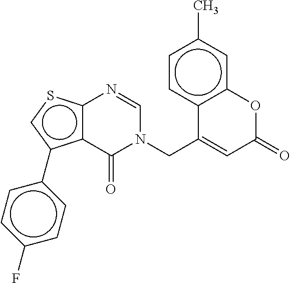 | 10.64 | >50.00 |
| AB00797753 | Enamine | T5990173 | 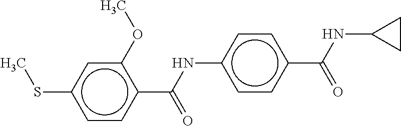 | 29.90 | >50.00 |
| AB00798716 | Enamine | T5978325 | 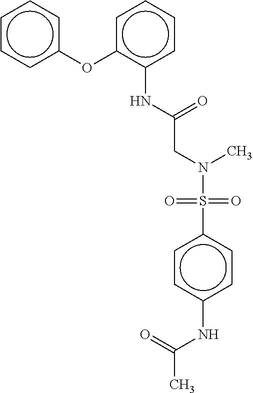 | 49.80 | >50.00 |
| AB00800399 | Enamine | T6034146 | 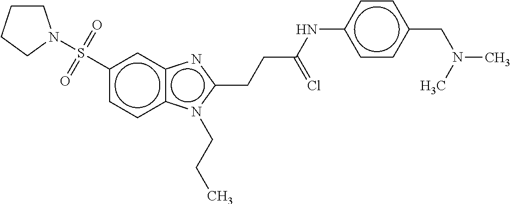 | 40.86 | >50.00 |
| AB00802246 | Enamine | T6013876 | 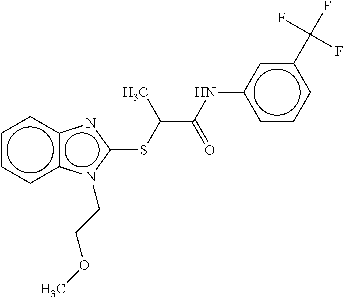 | 9.58 | >50.00 |
| AB00804772 | Enamine | T6045925 | 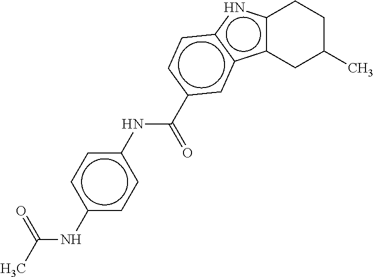 | 43.37 | >50.00 |
| AB00877180 | SRI Repository | SRI-23668 | 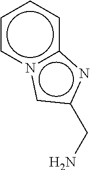 | 15.11 | >50.00 |
| AB00877365 | SRI Repository | SRI-23648 | 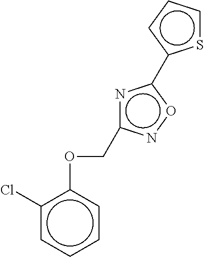 | 38.79 | >50.00 |
| AB00877398 | SRI Repository | SRI-23878 | 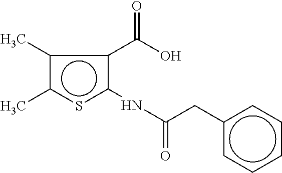 | 40.09 | >50.00 |
| AB00877405 | SRI Repository | SRI-23887 | 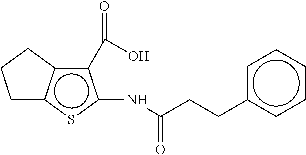 | 27.77 | >50.00 |
| SRI-101 | SRI Repository | SRI-101 | 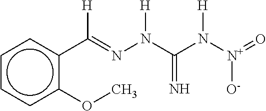 | 35.73 | >50.00 |
| SRI-10531 | SRI Repository | SRI-10531 | 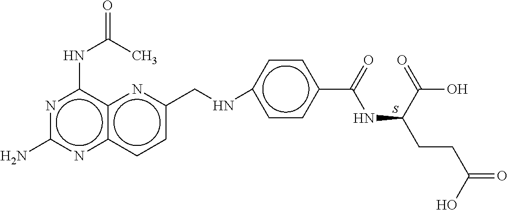 | 9.09 | >50.00 |
| SRI-4711 | SRI Repository | SRI-4711 | 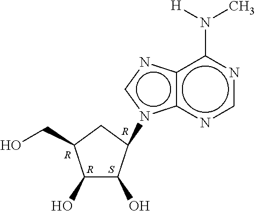 | 24.53 | >50.00 |
| SRI-7958 | SRI Repository | SRI-7958 | 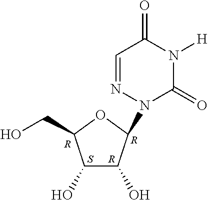 | 2.40 | >50.00 |

Virus Titer Reduction

To evaluate the effect of test compounds on the production of infectious progeny virus, a virus titer Reduction (VTR) assay was performed on test compounds confirmed in the HTS dose response assays. Four concentrations of each compound were evaluated (Table 3). Briefly, the four concentrations of test compounds, HEp-2 cells (8×10³ per well) and RSV, Long strain (MOI=0.9) were plated in 96 well microtiter plates. The plates were incubated for 48 hours. Fifteen (15) μl of 10-fold serial dilution of progeny virus containing medium from respective samples (test compound treated or untreated) was transferred to infect fresh HEp-2 cells (2×10³ cells per well) in a 384 well format. Plates were incubated for an additional 6 days and luminescence was measured using Cell Titer-Glo reagent (Promega Inc.) to determine cell viability. A well exhibiting equal to or greater than 50% cell viability was considered negative for virus infection. A well showing lesser than 50% cell viability was regarded as positive for virus infection. Four replicates for each virus dilution were analyzed and the number of virus positive wells (out of 4) was plotted versus the dilution. TCID₅₀ was calculated to be the dilution that produced two positive wells out of four using ActivityBase software (IDBS, Inc, Guilford, UK). The calculated TCID₅₀ value was used to further prioritize the scaffolds identified in the primary screen and confirmed in the dose response confirmatory assay.

TABLE 3
Test compound concentrations for VTR assay

| Library | Test Compound Concentrations | | | | | | | |
|---|---|---|---|---|---|---|---|---|
| Enamine | 50 | uM | 25 | uM | 12.5 | uM | 6.25 | uM |
| CB2 | 25 | ug/ml | 12.5 | ug/ml | 6.25 | ug/ml | 3.12 | ug/ml |
| SRI Repository | 50 | uM | 25 | uM | 12.5 | uM | 6.25 | uM |
| Ole Miss | 25 | uM | 12.5 | uM | 6.25 | uM | 3.12 | uM |
| SRI Collaborator | 30 | ug/ml | 15 | ug/ml | 7.5 | ug/ml | 3.75 | ug/ml |
| SRI Collaborator | 50 | ug/ml | 25 | ug/ml | 12. | ug/ml | 6.25 | ug/ml |

Three hundred and twelve active test compounds identified in the dose response assays (Table 2) were selected for titer reduction analysis. Forty-nine test compounds were identified based on the criteria of activity: more than 10 fold reduction in the progeny titer (>1 of log reduction) at 12 μg/ml and an efficacy EC₅₀ value equal to or less than 10 μg/ml (Table 4). There were at least 19 distinct chemotypes apparent in the 49 compounds screened by TCID₅₀.

TABLE 4

| | | | | | TCID- | TCID- | TCID- | TCID- |
|---|---|---|---|---|---|---|---|---|
| | | | | | 50 Re- | 50 Re- | 50 Re- | 50 Re- |
| | | | | | duction | duction | duction | duction |
| Compound | Sup- | Supplier | | | @ 3 | 6 @ | 12 @ | 25 @ |
| ID | plier | ID | Structure | | ug/ml | ug/ml | ug/ml | ug/ml |
| AB00289457 | Chem- bridge 2 | 6464487 | 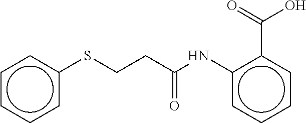 | | 1.00 | 4.90 | 6.73 | 6.57 |
| AB00308659 | Chem- bridge 2 | 7192352 | 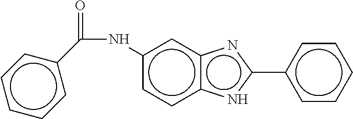 | | 2.82 | 3.64 | 6.43 | 6.40 |
| AB00313042 | Chem- bridge 2 | 7276383 | 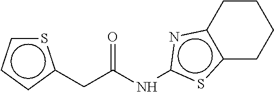 | | 3.56 | 5.24 | 5.43 | 6.40 |
| AB00369287 | Chem- bridge 2 | 7957878 | 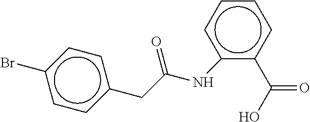 | | 1.00 | 3.31 | 4.73 | 6.57 |
| AB00359685 | Chem- bridge 2 | 7900141 | 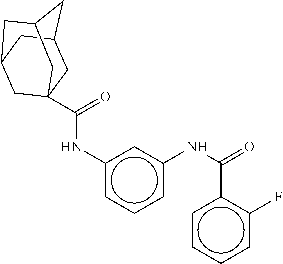 | | 1.00 | 2.13 | 4.40 | 4.94 |
| AB00279132 | Chem- bridge 2 | 5665705 | 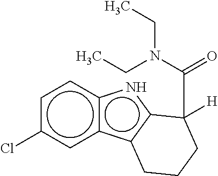 | | 1.00 | 3.63 | 4.22 | 3.83 |
| AB00280499 | Chem- bridge 2 | 5802791 | 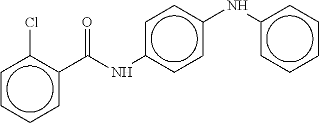 | | 2.55 | 2.38 | 3.69 | 2.96 |
| AB00353228 | Chem- bridge 2 | 7826467 | 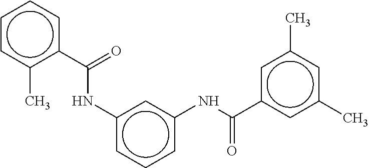 | | 1.55 | 2.87 | 3.42 | 3.43 |
| AB00369924 | Chem- bridge 2 | 7962703 | 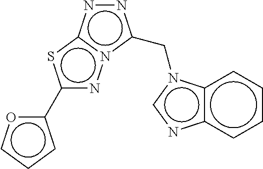 | | 1.00 | 2.62 | 3.22 | 2.61 |
| AB00370446 | Chem- bridge 2 | 7967599 | 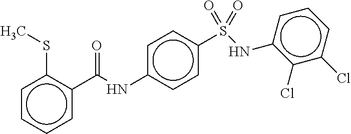 | | 1.00 | 1.32 | 3.21 | 6.57 |
| AB00345103 | Chem- bridge 2 | 7767847 | 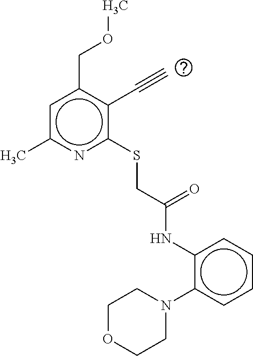 | | 1.00 | 1.31 | 3.20 | 2.86 |
| AB00361531 | Chem- bridge 2 | 7908294 | 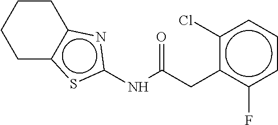 | | 1.55 | 1.74 | 3.09 | 3.78 |
| AB00298501 | Chem- bridge 2 | 6980267 | 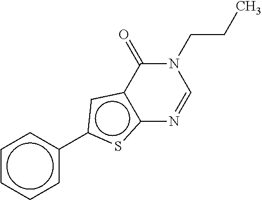 | | 1.82 | 2.38 | 2.90 | 3.78 |
| AB00296320 | Chem- bridge 2 | 6944380 | 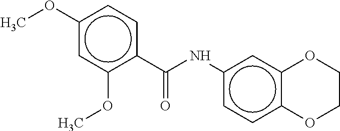 | | 1.55 | 1.38 | 2.90 | 4.66 |
| AB00362846 | Chem- bridge 2 | 7915886 | 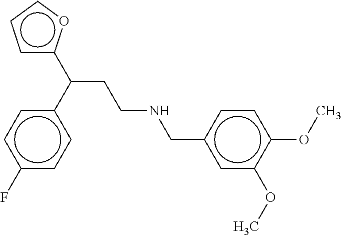 | | 1.00 | 1.92 | 2.82 | 5.48 |
| AB00302018 | Chem- bridge 2 | 7020237 | 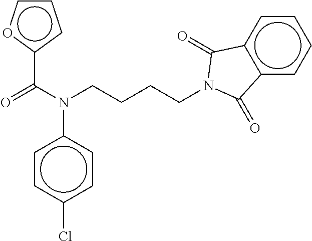 | | 1.66 | 2.64 | 2.68 | 3.07 |
| AB00312102 | Chem- bridge 2 | 7259967 | 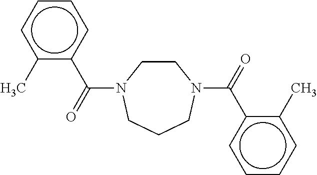 | | 1.53 | 1.86 | 2.68 | 2.39 |
| AB00367930 | Chem- bridge 2 | 7949301 | 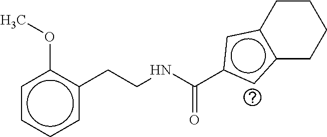 | | 1.82 | 1.04 | 2.68 | 6.40 |
| AB00356285 | Chem- bridge 2 | 7853019 | 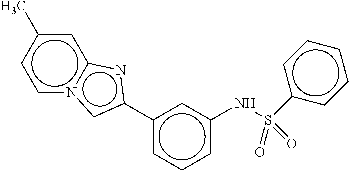 | | 1.14 | 1.38 | 2.42 | 3.78 |
| AB00341850 | Chem- bridge 2 | 7735547 | 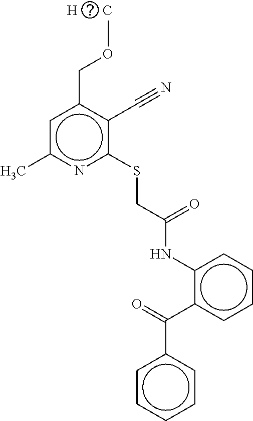 | | 1.00 | 2.30 | 2.41 | 2.23 |
| AB00275559 | Chem- bridge 2 | 5161544 | 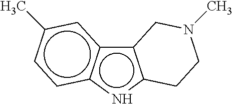 | | 1.00 | 1.93 | 2.21 | 1.93 |
| AB00355020 | Chem- bridge 2 | 7844977 | 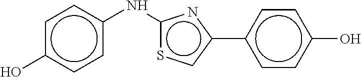 | | 1.00 | 1.01 | 2.20 | 2.55 |
| AB00285095 | Chem- bridge 2 | 6133629 | 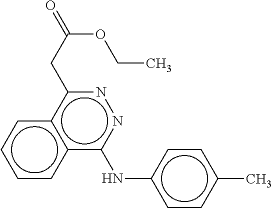 | | 1.00 | 1.32 | 1.99 | 2.56 |
| AB00359299 | Chem- bridge 2 | 7889870 | 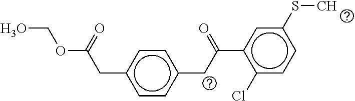 | | 1.00 | 1.22 | 1.99 | 2.83 |
| AB00301184 | Chem- bridge 2 | 7012545 | 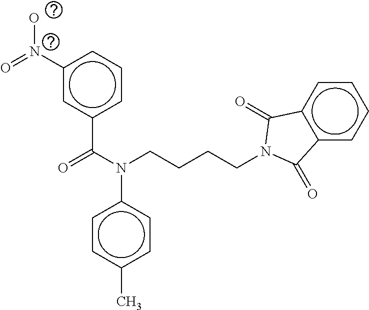 | | 1.53 | 1.67 | 1.90 | 1.88 |
| AB00310739 | Chem- bridge 2 | 7236979 | 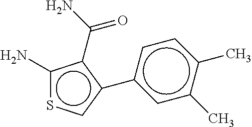 | | 1.03 | 1.86 | 1.90 | 2.39 |
| AB00311298 | Chem- bridge 2 | 7247789 | 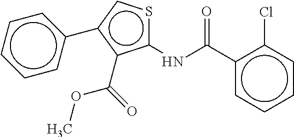 | | 1.53 | 1.86 | 1.90 | 1.88 |
| AB00368252 | Chem- bridge 2 | 7951033 | 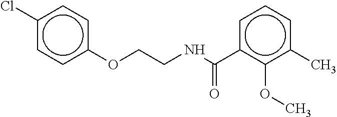 | | 1.00 | 1.30 | 1.72 | 4.94 |
| AB00370331 | Chem- bridge 2 | 7966004 | 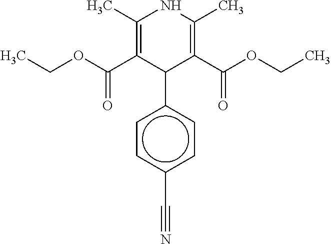 | | −0.15 | 1.39 | 1.70 | 2.89 |
| AB00306112 | Chem- bridge 2 | 7135263 | 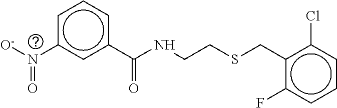 | | 1.85 | 1.38 | 1.68 | 2.88 |
| AB00280244 | Chem- bridge 2 | 5790700 | 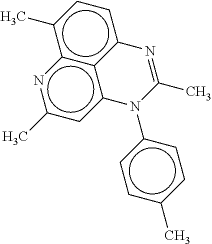 | | 1.55 | 1.36 | 1.55 | 2.89 |
| AB00301782 | Chem- bridge 2 | 7017760 | 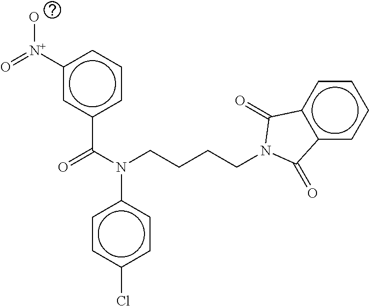 | | 1.55 | 1.64 | 1.42 | 0.87 |
| AB00309154 | Chem- bridge 2 | 7204366 | 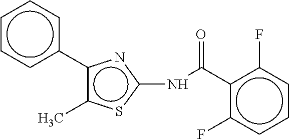 | | 1.54 | 1.38 | 1.42 | 1.88 |
| AB00314111 | Chem- bridge 2 | 7310127 | 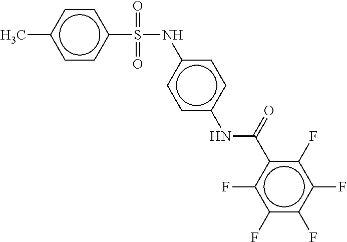 | | 1.00 | 1.01 | 1.41 | 3.05 |
| AB00276073 | Chem- bridge 2 | 5231477 | 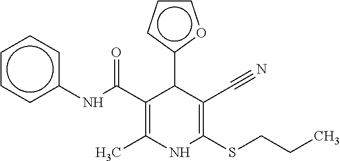 | | 1.00 | 0.63 | 1.39 | 3.57 |
| | | | | TCID- | TCID- | TCID- | TCID- | TCID- |
| | | | | 50 Re- | 50 Re- | 50 Re- | 50 Re- | 50 Re- |
| | | | | duction | duction | duction | duction | duction |
| Compound | Sup- | Supplier | | @ 3 | @ 5 | @ 12 | @ 25 | @ 50 |
| ID | plier | ID | Structure | ug/ml | ug/ml | ug/ml | ug/ml | ug/ml |
| AB00174524 | SRI Repos- itory | SRI- 10013 | 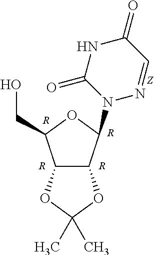 | | 4.58 | 5.98 | 5.56 | 5.87 |
| SRI-7958 | SRI Repos- itory | SRI- 7958 | 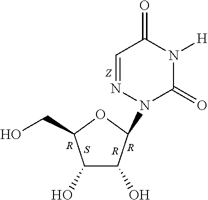 | | 3.58 | 5.98 | 5.56 | 5.87 |
| AB00311948 | Chem- bridge 2 | 7257552 | 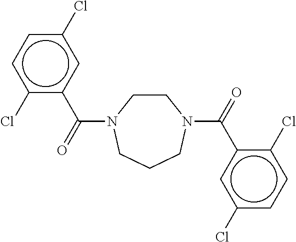 | 3.13 | 4.64 | 5.56 | 5.90 | |
| AB00318114 | Chem- bridge 2 | 7396766 | 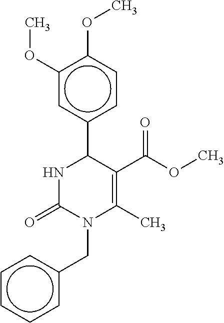 | 2.42 | 4.69 | 5.56 | 5.90 | |
| AB00725538 | Ena- mine | T5259183 | 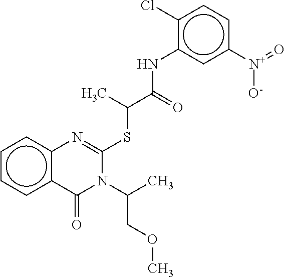 | | 2.95 | 4.98 | 5.56 | 5.87 |
| AB00319298 | Chem- bridge 2 | 7420864 | 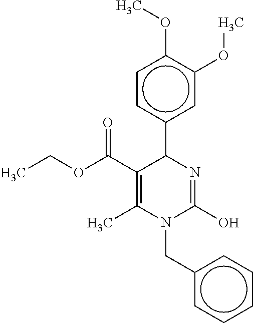 | 0.84 | 3.09 | 4.56 | 5.90 | |
| AB00785138 | Ena- mine | T5949368 | 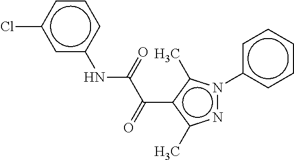 | | 1.96 | 4.24 | 4.56 | 3.87 |
| AB00705281 | SRI Colla- borator | J5063 | 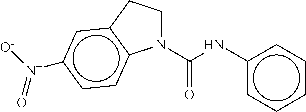 | | 4.75 | 4.20 | 5.56 | 5.90 |
| AB00802246 | En- amine | T6013876 | 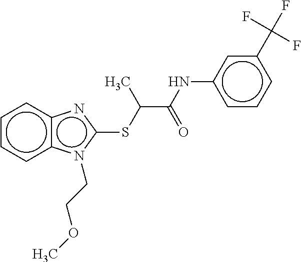 | | 2.26 | 3.98 | 4.42 | 5.87 |
| AB00292352 | Chem- bridge 2 | 6663053 | 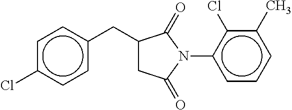 | 1.22 | 2.31 | 3.82 | 5.90 | |
| AB00514453 | SRI Repos- itory | SRI- 19094 | 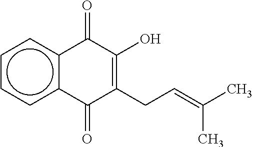 | | 0.06 | 3.33 | 4.47 | 4.87 |
| AB00296415 | Chem- bridge 2 | 6944917 | 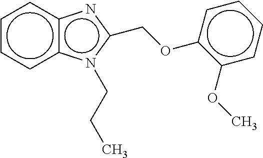 | 1.74 | 2.48 | 2.59 | 5.90 | |
| AB00300194 | Chem- bridge 2 | 7004218 | 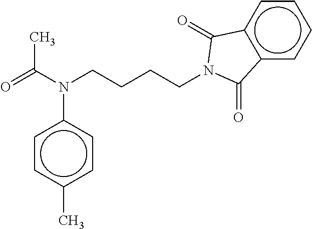 | 1.41 | 1.83 | 2.04 | 2.56 | |
| SRI-10531 | SRI Repos- itory | SRI- 10531 | 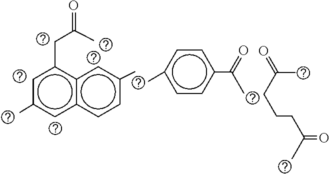 | | 0.16 | 0.97 | −0.02 | 0.28 |

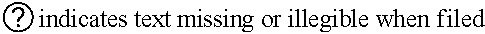

Many of the compounds described in the Tables above are commercially available. Compounds that are not commercially available can be readily synthesized by those skilled in the art. Furthermore, the compounds of the instant disclosure can be prepared according to the exemplary procedures described below.

Compounds of formula 5 can be prepared using the methodology described in International Patent Application Publication No. WO 2009/016119 to Wagner et al., entitled “Substituted N-Heterocyclic Arylsulfonylaminomethylphosphonic Acid Derivatives as Medicaments for Treatment of Type I and II Diabetes Mellitus and Process for their Preparation,” which is incorporated herein by reference in its entirety.

Compounds of formula 9 can be prepared according to the general procedure set forth below in Example 1 for synthesis of SRI 10531.

### Example 1

### Synthesis of SRI 10531

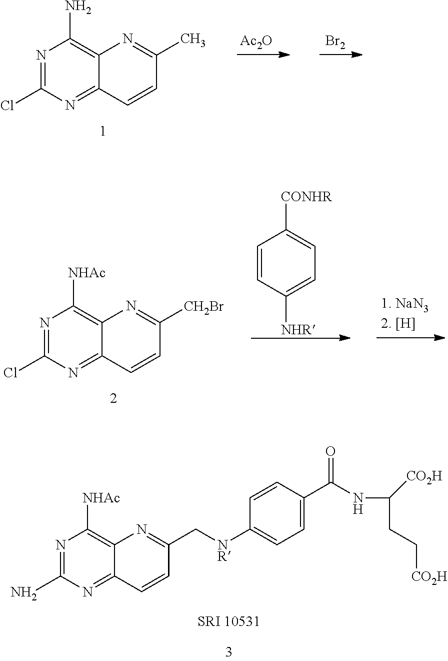

A. Compound 1 (4.0 g) was treated with acetic anhydride (80 mL) at 150° C. for 30 min. The solvent was evaporated and the residue purified by a silica gel column to give a colorless solid intermediate (3.27 g).

B. To a solution of the intermediate from step (A) (1.0 g) in 200 mL of anhydrous CCl₄, 0.2 mL of bromine was added and the solution was refluxed for 4 hours. The solvent was removed and the residue was purified on a column to give 1.17 g of compound 2 as a colorless solid.

C. To a solution of 1.869 g of 4-aminobenzoyl-L-glutamic acid in 10 mL of N,N-dimethylacetamide was added 1.60 g of 2.

The mixture was stirred at room temperature for 48 h. The solvent was evaporated and the residue was purified via column chromatography over silica gel to give a colorless solid (0.82 g). This material (100 mg) in 5 mL of N,N-dimethylacetamide was treated with NaN₃ (100 mg). The mixture was stirred at 95-100° C. for 5 h. The solution was then concentrated and the residue purified on a column, followed by catalytic hydrogenation (Pd—C in ethanol, H₂, 20 psi) to afford SRI 10531 as a colorless solid (65 mg).

Compounds of formula 11 can be prepared using the methodology described in U.S. Patent Application Publication No. 20030187040 to Pevarello et al., entitled “2-Ureidothiazole Derivatives, Process for Their Preparation, and Their Use as Antitumor Agents. Patent US 20030187040, which is incorporated herein by reference in its entirety See, e.g., example 16, Preparation of Imidazo[1,2-a]pyridin-2-ylmethanamine.

Compounds of formula 13 can be prepared according to the general procedure set forth below in Example 2 for synthesis of AB00700560.

### Example 2

### Synthesis of AB00700560

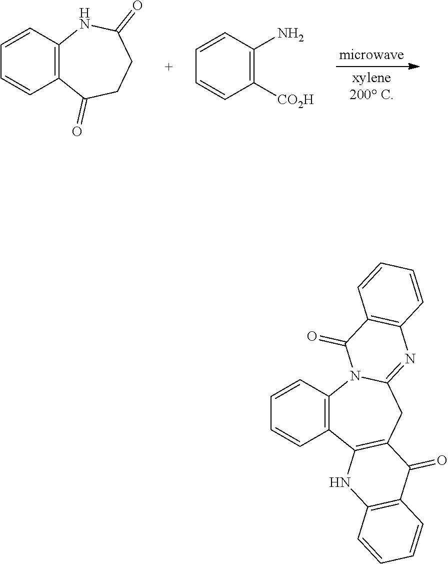 [SMILES: O=C1CCC(=O)c2ccccc2N1 ; Nc1ccccc1C(=O)O ; O=c1c2c([nH]c3ccccc13)-c1ccccc1-n1c(nc3ccccc3c1=O)C2]

A mixture of 3,4-dihydro-1H-1-benzazepine-2,5-dione (0.175 g, 1.0 mmol) and anthranilic acid (0.15 g, 2.2 mmol) in a 12 mL quartz reaction vessel was irradiated in a focused monomode microwave reactor at 200° C. for 10 min. The reaction mixture was cooled, treated with water and the mixture was basified with conc. aqueous NH₄OH. The mixture was then extracted with CHCl₃, dried with anhydrous Na₂SO₄, filtered, and the filtrate was concentrated to dryness under reduced pressure. The residue obtained was purified by chromatography over a column of silica using 4% MeOH in CHCl₃ as the eluent to obtain 0.07 g (18% yield) of the product.

Compounds of formula 14, can be prepared using standard nucleoside coupling reactions, for example, by coupling 6-azauracil (commercially available) or its analog to 1-O-benzyl-2,3,4-triacetylribose. Compounds of formula 14 can be prepared according to the general procedure set forth below in Example 3 for synthesis of AB00174524.

### Example 3

### Synthesis of AB00174524

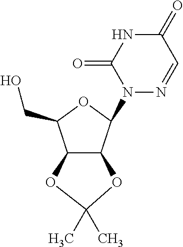 [SMILES: CC1(C)O[C@@H]2[C@H](O1)[C@H](n1ncc(=O)[nH]c1=O)O[C@@H]2CO]

6-Azauridine (5.0 g) and copper sulphate (anhydrous, 10 g) were suspended in 125 mL of anhydrous acetone in an atmosphere of dry argon. To the mixture 0.13 mL of concentrated sulfuric acid was added and stirred for 64 hours at room temperature. The mixture was filtered. To the filtrate was added ammonia/methanol (2 ml), and the solution was evaporated. The residue was recrystallized from ethyl acetate and dried in vacuo over P₂O₅ to give the desired product as a colorless solid (3.2 g).

Compounds of formula 15, can be prepared using standard nucleoside coupling reactions, for example, by coupling triazolylformamide (commercially available) or its analog to 1-O-benzyl-2,3,4-triacetylribose. Compounds of formula 15 can be prepared according to the general procedure set forth above in Example 3.

Compounds of formula 17 can be prepared using the methodology described in Ouyang et al., Synthesis and Fluorescent Properties of 2-(1H-Benzimidazol-2-yl)-phenol Derivatives, JOURNAL OF HETEROCYCLIC CHEMISTRY 41(3): 359-65 (2004), which is incorporated herein by reference in its entirety.

### Formulations

The compounds of the present disclosure can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice. The compounds can also be administered in conjunction with other therapeutic agents.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this disclosure can be administered by any conventional method available for use in conjunction with pharmaceuticals, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg.

Dosage forms (compositions suitable for administration) typically contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present disclosure should be sufficient to affect a therapeutic response in the animal over a reasonable time frame. One skilled in the art will recognize that dosage will depend upon a variety of factors including a condition of the animal, the body weight of the animal, as well as the severity and stage of the condition being treated.

A suitable dose is that which will result in a concentration of the active agent in a patient which is known to affect the desired response. The preferred dosage is the amount which results in maximum inhibition of the condition being treated, without unmanageable side effects.

The size of the dose also will be determined by the route, timing and frequency of administration as well as the existence, nature, and extend of any adverse side effects that might accompany the administration of the compound and the desired physiological effect.

Useful pharmaceutical dosage forms for administration of the compounds according to the present disclosure can be illustrated as follows:

### Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

### Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

### Tablets

A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

### Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present disclosure can be administered in the form of nose drops, or metered dose and a nasal or buccal inhaler. The drug is delivered from a nasal solution as a fine mist or from a powder as an aerosol.

All publications, patents and patent applications cited in this specification are herein incorporated by reference, and for any and all purpose, as if each individual publication, patent or patent application were specifically and individually indicated to be incorporated by reference. In the case of inconsistencies, the present disclosure will prevail.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The embodiments described hereinabove are further intended to explain best modes known of practicing it and to enable others skilled in the art to utilize the disclosure in such, or other, embodiments and with the various modifications required by the particular applications or uses. Accordingly, the description is not intended to limit it to the form disclosed herein. Also, it is intended that the appended claims be construed to include alternative embodiments.

What is claimed is:

1. A method for treating a human infected with a virus by administering to said human an effective amount of at least one compound represented by the formulas 16, 4 6, 1, 7, 17, 5, 3, 2, 18, 19, 11, 9, 8, 10, 12, 13, 14 and 15 below: 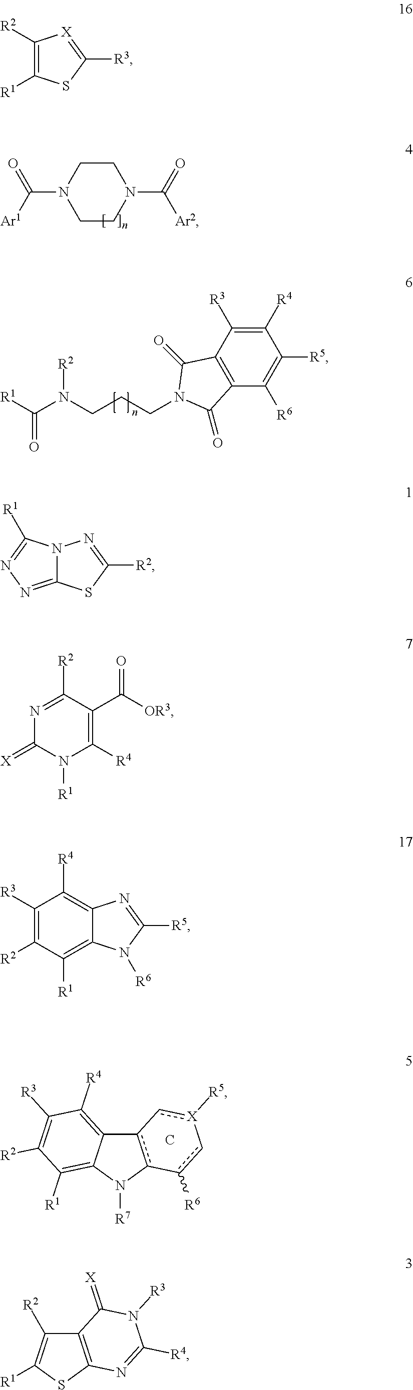 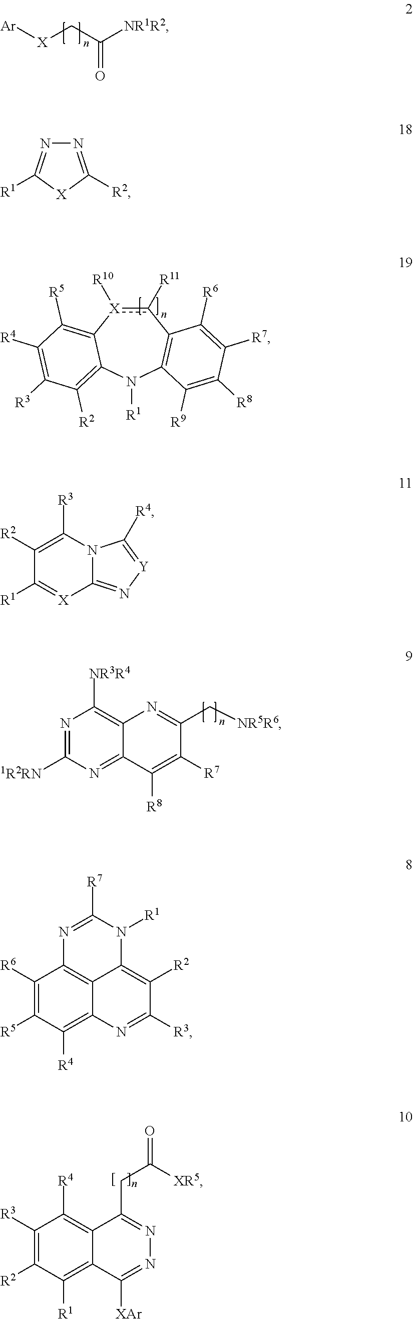 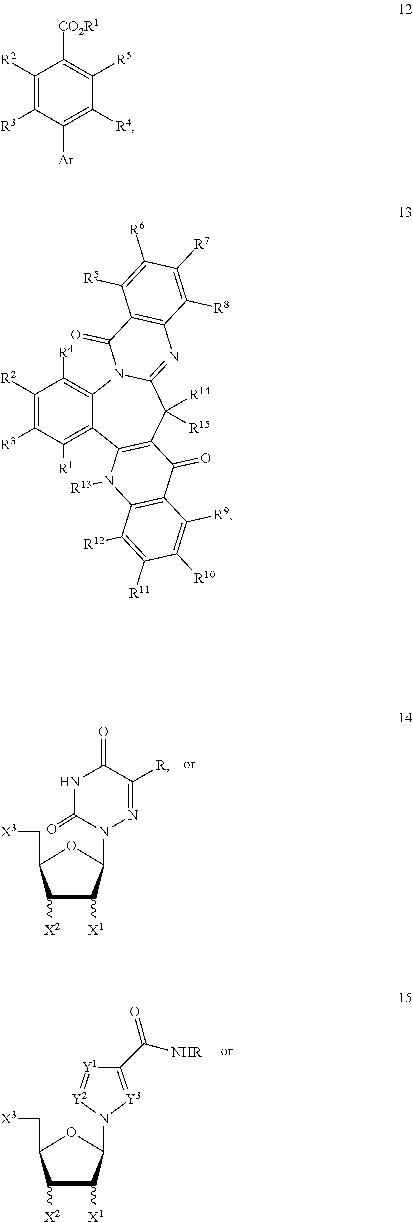
a pharmaceutically acceptable salt thereof, a solvate thereof, or a prodrug thereof;
wherein in formula 16,
X is chosen from the group consisting of N and C;
R¹, R², and R³ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
if X═C, it may be further independently substituted by a group from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 4,
n=0, 1, or 2.
Ar¹ may be attached directly to the nitrogen atom without the linking carbonyl group and if both carbonyl groups are present, then one of the nitrogen atoms may be replaced by carbon;
Ar¹ and Ar² are each independently chosen from the group consisting of substituted or unsubstituted aryl, substituted or unsubstituted arylalkyl, substituted or unsubstituted heteroaryl, substituted or unsubstituted cycloalkyl, and substituted or unsubstituted heterocyclic;
wherein in formula 6,
n=1, 2, or 3;
R¹ is chosen from the group consisting of substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, alkylthio, amino, and mono- or di-substituted amino;
R² is chosen from the group consisting of hydrogen, substituted or unsubstituted alkyl, substituted or unsubstituted unsaturated alkyl, substituted or unsubstituted aryl; substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R³-R⁶ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 1,
each R¹ and R² is independently chosen from the group consisting of hydrogen, substituted or unsubstituted alkyl, substituted or unsubstituted alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate ester, carboxamido, amino, and mono- or di-substituted amino;
wherein in formula 7,
X is chosen from oxygen and sulfur;
R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic including glycosyl rings, acyl, carboxylate esters, and carboxamido;
R² and R⁴ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;
wherein in formula 17,
R¹ through R⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R⁶ is chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
wherein in formula 5,
the ring designated C is optional but if present may be saturated or partially or fully unsaturated;
if ring C is absent, then the pyrrole ring may optionally have one or two additional substituents instead;
X is substituted or unsubstituted carbon or substituted or unsubstituted nitrogen;
R¹-R⁴, R⁶, and R⁵ when X═C, are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
when ring C is absent, then the one or two additional substituents on the pyrrole ring, if present, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R⁵ when X═N, and R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl; carboxyl, carboxylate esters, and carboxamido;
wherein in formula 3,
X is chosen from oxygen or substituted nitrogen,
R¹, R², and R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxyl, carboxylate esters, and carboxamido;
wherein in formula 2,
n=1, 2, or 3;
X is chosen from sulfur, oxygen, and substituted or unsubstituted nitrogen;
Ar is chosen from the group consisting of substituted or unsubstituted aryl, substituted or unsubstituted arylalkyl, substituted or unsubstituted heteroaryl, substituted or unsubstituted cycloalkyl, or substituted or unsubstituted heterocyclic;
R¹ and R² are independently chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, and acyl;
wherein in formula 18,
X is chosen from the group consisting of O, S, and N;
R¹ and R² are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
if X═N, then it is substituted by a moiety chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
wherein in formula 19,
n=0 or 1;
if n=0, the substituent R¹¹ does not occur;
X is chosen from the group consisting of C, O, S, and N;
if X═C or X═N, then it may substituted by a group R¹⁰;
if X═O or X═S, then substituent R¹⁰ does not occur;
if n=1 and X═C or N, then the bond to X internal to the seven-membered ring may optionally be unsaturated;
R¹ and R¹⁰ in the case X═N, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R² through R⁹, R¹⁰ in the case X═C, and substituent R¹¹ in the case n=1, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.
wherein in formula 11,
X and Y are independently chosen from C and N;
R¹ through R⁴ are independently chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
if X═C or Y═C, then either may independently be substituted by additional R moieties chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 9,
n=0, 1, or 2;
R¹ through R⁶ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R⁷ and R⁸ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 8,
R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R² through R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 10,
n=0, 1, or 2;
X is chosen from the group consisting of N, O, and S;
R¹ through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R⁵ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic;
wherein in formula 12,
R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;
R² through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic;
wherein in formula 13,
R¹ through R¹², R¹⁴ and R¹⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R¹³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
wherein in formula 14,
X¹, X², and X³ are independently chosen from the group consisting of O, S, N, C, and halogen;
whenever any of X¹, X², and X³ is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;
the R group is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 15,
X¹, X², and X³ are independently chosen from the group consisting of O, S, N, C, and halogen;
whenever any of these groups is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;
Y¹, Y², and Y³ are independently chosen from the group consisting of N and C;
R is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido.

2. The method of claim 1 wherein the virus is a respiratory virus.

3. The method of claim 1 wherein the virus is selected from the group consisting of the families Paramyxoviridae, human metapneumovirus, human parainfluenza virus, measles virus, and mumps virus.

4. The method of claim 1 wherein the virus is respiratory syncytial virus.

5. A method for preventing virus-induced cytopathic effect (CPE) in a human patient for protection against infections which comprises administering to said human an effective amount of at least one compound represented by formulas 16, 4, 6, 1, 7, 17, 5, 3, 2, 18, 19, 11, 9, 8, 10, 12, 13, 14 and 15, 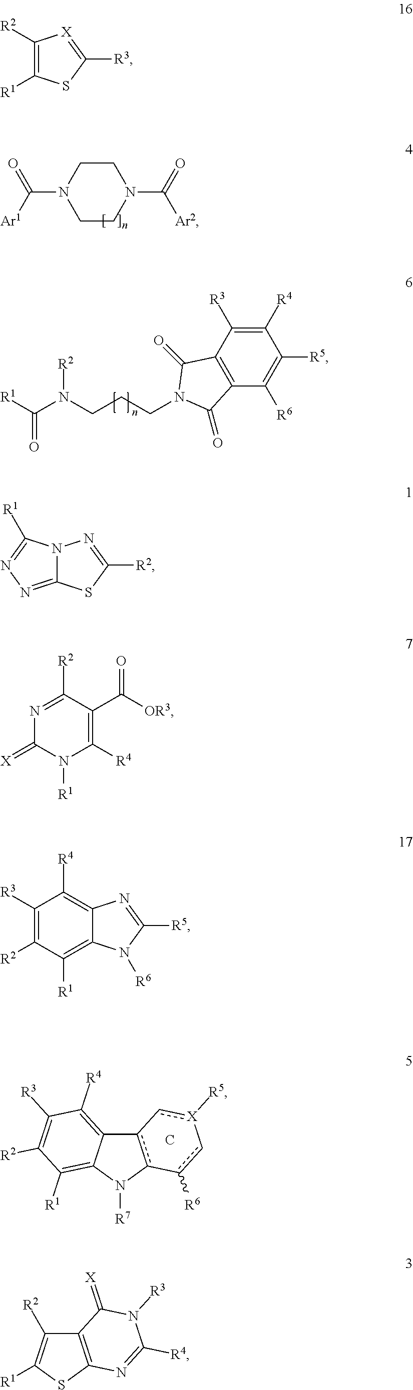 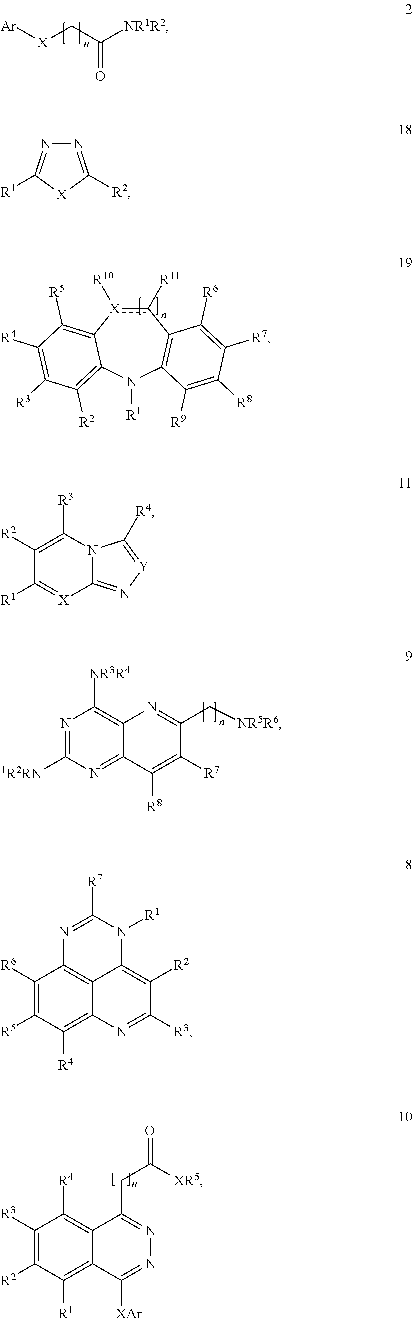 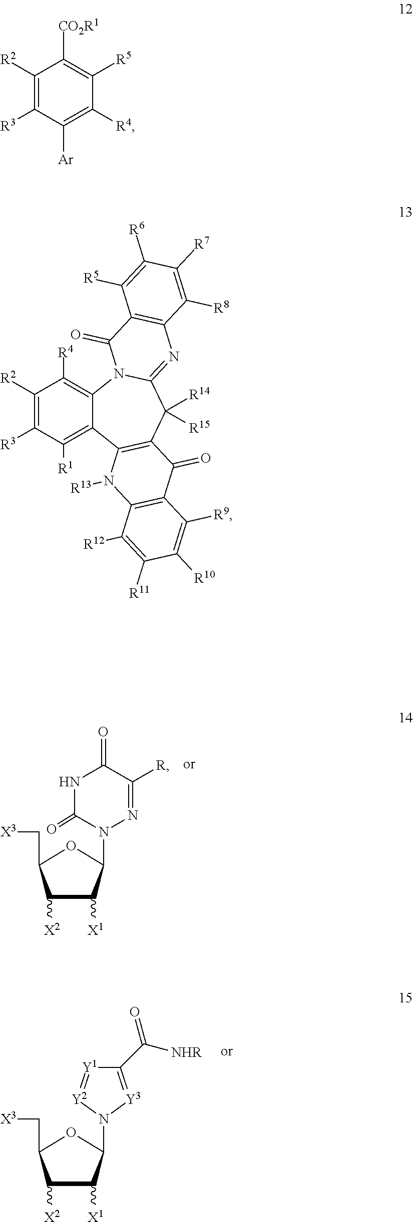
a pharmaceutically acceptable salt thereof, a solvate thereof, or a prodrug thereof;
wherein in formula 16,
X is chosen from the group consisting of N and C;
R¹, R², and R³ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
if X═C, it may be further independently substituted by a group from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 4,
n=0, 1, or 2;
Ar¹ may be attached directly to the nitrogen atom without the linking carbonyl group and if both carbonyl groups are present, then one of the nitrogen atoms may be replaced by carbon;
Ar¹ and Ar² are each independently chosen from the group consisting of substituted or unsubstituted aryl, substituted or unsubstituted arylalkyl, substituted or unsubstituted heteroaryl, substituted or unsubstituted cycloalkyl, and substituted or unsubstituted heterocyclic;
wherein in formula 6,
n=1, 2, or 3;
R¹ is chosen from the group consisting of substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, alkylthio, amino, and mono- or di-substituted amino;
R² is chosen from the group consisting of hydrogen, substituted or unsubstituted alkyl, substituted or unsubstituted unsaturated alkyl, substituted or unsubstituted aryl; substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R³-R⁶ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 1,
each R¹ and R² is independently chosen from the group consisting of hydrogen, substituted or unsubstituted alkyl, substituted or unsubstituted alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate ester, carboxamido, amino, and mono- or di-substituted amino;
wherein in formula 7,
X is chosen from oxygen and sulfur;
R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic including glycosyl rings, acyl, carboxylate esters, and carboxamido;
R² and R⁴ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;
wherein in formula 17,
R¹ through R⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R⁶ is chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
wherein in formula 5,
the ring designated C is optional but if present may be saturated or partially or fully unsaturated;
if ring C is absent, then the pyrrole ring may optionally have one or two additional substituents instead;
X is substituted or unsubstituted carbon or substituted or unsubstituted nitrogen;
R¹-R⁴, R⁶, and R⁵ when X═C, are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
when ring C is absent, then the one or two additional substituents on the pyrrole ring, if present, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R⁵ when X═N, and R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl; carboxyl, carboxylate esters, and carboxamido;
wherein in formula 3,
X is chosen from oxygen or substituted nitrogen,
R¹, R², and R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxyl, carboxylate esters, and carboxamido;
wherein in formula 2,
n=1, 2, or 3;
X is chosen from sulfur, oxygen, and substituted or unsubstituted nitrogen;
Ar is chosen from the group consisting of substituted or unsubstituted aryl, substituted or unsubstituted arylalkyl, substituted or unsubstituted heteroaryl, substituted or unsubstituted cycloalkyl, or substituted or unsubstituted heterocyclic;
R¹ and R² are independently chosen from the group consisting of hydrogen, substituted or unsubstituted alkyl, substituted or unsubstituted unsaturated alkyl; substituted or unsubstituted aryl, substituted or unsubstituted heteroaryl, substituted or unsubstituted arylalkyl, substituted or unsubstituted cycloalkyl, substituted or unsubstituted heterocyclic, and acyl;
wherein in formula 18,
X is chosen from the group consisting of O, S, and N;
R¹ and R² are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
if X═N, then it is substituted by a moiety chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
wherein in formula 19,
n=0 or 1;
if n=0, the substituent R¹¹ does not occur;
X is chosen from the group consisting of C, O, S, and N;
if X═C or X═N, then it may substituted by a group R¹⁰;
if X═O or X═S, then substituent R¹⁰ does not occur;
if n=1 and X═C or N, then the bond to X internal to the seven-membered ring may optionally be unsaturated;
R¹ and R¹⁰ in the case X═N, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R² through R⁹, R¹⁰ in the case X═C, and substituent R¹¹ in the case n=1, are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino.
wherein in formula 11,
X and Y are independently chosen from C and N;
R¹ through R⁴ are independently chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
if X═C or Y═C, then either may independently be substituted by additional R moieties chosen from the group consisting of hydrogen; substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 9,
n=0, 1, or 2;
R¹ through R⁶ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R⁷ and R⁸ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 8,
R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
R² through R⁷ are each independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 10,
n=0, 1, or 2;
X is chosen from the group consisting of N, O, and S;
R¹ through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R⁵ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic;
wherein in formula 12,
R¹ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, and substituted or unsubstituted heterocyclic;
R² through R⁴ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
Ar is chosen from the group consisting of hydrogen, substituted or unsubstituted aryl, substituted or unsubstituted heteroaryl, substituted or unsubstituted arylalkyl, substituted or unsubstituted cycloalkyl, and substituted or unsubstituted heterocyclic;
wherein in formula 13,
R¹ through R¹², R¹⁴ and R¹⁵ are independently chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
R¹³ is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
wherein in formula 14,
X¹, X², and X³ are independently chosen from the group consisting of O, S, N, C, and halogen;
whenever any of X¹, X², and X³ is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;
the R group is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, hydroxy, alkoxy, carbonyloxy, halogen, azido, cyano, nitro, alkylthio, carboxyl, carboxylate esters, carboxamido, amino, and mono- or di-substituted amino;
wherein in formula 15,
X¹, X², and X³ are independently chosen from the group consisting of O, S, N, C, and halogen;
whenever any of these groups is not halogen, they may be independently further substituted from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido;
whenever any of X¹, X², and X³ is C, they may additionally be substituted from the group consisting of hydroxy, alkoxy, carbonyloxy, halogen, azido, cyano, nitro, alkylthio, carboxyl, amino, and mono- or di-substituted amino;
Y², and Y³ are independently chosen from the group consisting of N and C;
R is chosen from the group consisting of hydrogen, substituted or unsubstituted alkyl, substituted or unsubstituted unsaturated alkyl, substituted or unsubstituted aryl, substituted or unsubstituted heteroaryl, substituted or unsubstituted arylalkyl, substituted or unsubstituted cycloalkyl, substituted or unsubstituted heterocyclic, acyl, carboxylate esters, and carboxamido.

6. The method of claim 5 wherein the virus is a respiratory virus.

7. The method of claim 5 wherein the virus is selected from the group consisting of the families Paramyxoviridae, human metapneumovirus, human parainfluenza virus, measles virus, and mumps virus.

8. The method of claim 5 wherein the virus is respiratory syncytial virus.

9. A method for screening for compounds for use as an anti-viral agent against a virus which comprises obtaining frozen cells infected with said virus, thawing said infected cells and mixing said infected cells with uninfected cells of the same type as the infected cells, contacting the mixture of said infected cells and uninfected cells with a compound to be screened and determining the viability of said cells.

10. The method of claim 9 wherein the virus is a respiratory virus.

11. The method of claim 9 wherein the virus is selected from the group consisting of the families Paramyxoviridae, human metapneumovirus, human parainfluenza virus, measles virus, and mumps virus.

12. The method of claim 9 wherein the virus is respiratory syncytial virus.

13. The method of claim 9 wherein said cells are HEp-2 cells.

14. The method of claim 9 wherein the infected cells are admixed with uninfected cells in a weight ratio of 1:100.

15. The method of claim 1, which comprises administering to said human an effective amount of at least one compound represented by the formula 16.

16. The method of claim 1, which comprises administering to said human an effective amount of at least one compound represented by the formula 16.
